(19) United States
(12) Patent Application Publication
(10) Pub. No.: US 2022/0403414 A1
(43) Pub. Date: Dec. 22, 2022

(54) NOVEL AAV VARIANT

(71) Applicant: WUXI APPTEC (SHANGHAI) CO., LTD., Shanghai (CN)

(72) Inventors: Qunsheng JI, Shanghai (CN); Yuan LU, Shanghai (CN); Qing LIN, Shanghai (CN); Yixiong CHEN, Shanghai (CN)

(21) Appl. No.: 17/636,324

(22) PCT Filed: Oct. 15, 2020
(86) PCT No.: PCT/CN2020/121099

§ 371 Date: Feb. 17, 2022

(30) Foreign Application Priority Data
Oct. 16, 2019 (CN) PCT/CN2019/111525

(51) Int. Cl. C12N 15/86; A61K 48/00

(52) U.S. Cl. CPC C12N 15/86; A61K 48/0058; C12N 2750/14122; C12N 2750/14123; C12N 2750/14142

(57) ABSTRACT

A variant AAV capsid protein, comprising an amino acid sequence corresponding to the position amino acids 585 to 597 or 598 of AAV8 or the position amino acids 583 to 595 or 596 of AAV9, and the polynucleotide, host cells, vectors, AAV virion or the pharmaceutical composition thereof. A method of treating disease, the method comprising administering to a subject in need thereof an effective amount of the recombinant AAV virion.

### CROSS-REFERENCE TO RELATED APPLICATIONS

This application claims the benefit of and priority to PCT Application No. PCT/CN2019/111525, filed Oct. 16, 2019, the entire contents of which are incorporated by reference herein for all purposes.

### TECHNICAL FIELD

The present invention relates to gene therapy, especially refers to adeno-associated virus (AAV).

### BACKGROUND OF THE INVENTION

Clinical gene therapy has been increasingly successful owing both to an improving understanding of human disease and to breakthroughs in gene delivery technologies. Among these technologies, recombinant adeno-associated viral (rAAV) vectors are being used in a growing number of clinical trials. rAAV offers several important advantages: (1) Favorable safety profile: no known human disease is associated with AAV. The virus can only replicate under special circumstances. There is no viral protein in the vector. The rAAV vector rarely integrates into the host genome. (2) Ability to infect both dividing and non-dividing cells in vitro and in vivo. (3) Long-term expression of the transgene in various tissues. (4) Absence of enormous immune response. All of them contribute to proven efficacy in numerous animal models, as well as increasing number of clinical studies including Leber's congenital amaurosis, Hemophilia B, Alpha-1 antitrypsin deficiency, Parkinson's disease, Canavan's disease and muscular dystrophies. In 2012, the European Commission approved a rAAV-based product for lipoprotein lipase deficiency, the first gene therapy product approved by the western world. In 2017, voretigene neparvovec-rzyl (Luxturna), a gene therapy used to treat biallelic RPE65 mutations-associated retinal dystrophy marked the first US Food and Drug Administration (FDA) approved in vivo gene therapy product. On May 24, 2019, the US FDA approved the first drug for spinal muscular atrophy, which is also an AAV-based gene therapy.

Challenges of gene delivery using AAV vector arise from the simple consideration that the properties that allow AAV natural infection are distinct from those needed for most medical applications such as gene therapy. As a result, AAV vector engineering such that viruses can release from the constraints of natural evolution and thereby enable them to acquire novel and biomedically valuable phenotypes has been and will continue to be a major challenge in the research field.

With an increasing number of clinical stage gene therapy studies, AAV8 and AAV9, another two naturally-occurring serotypes, have demonstrated more powerful gene delivery capability. However, the primary cellular receptor for AAV8 and AAV9 remain unknown. At present, the engineering of AAV8 and AAV9 vectors for both basic understanding as well as gene delivery applications are limited.

### SUMMARY

The present invention provides a variant AAV capsid protein, comprising one or more amino acid substitutions, the capsid protein comprises a substituted amino acid sequence corresponding to VR VIII region of the native AAV 8 or AAV9 capsid protein.

In one embodiment, the variant AAV capsid protein comprises a variant AAV capsid protein comprising a substitution at one or more of amino acid residues N585, L586, Q587, Q588, Q589, N590, T591, A592, P593, Q594, 1595, G596, T597, V598, corresponding to amino acid sequence of the native AAV 8 (SEQ ID NO:1), the substitution of amino acid residues is selected from N585Y, L586N, L586Q, L586K, L586H, L586F, Q587N, Q588 N, Q588S, Q588A, Q588D, Q588G, Q589T, Q589A, Q589G, Q589S, Q589N, N590A, N590S, N590D, N590T, N590Q, T591S, T591A, T591R, T591E, T591G, A592Q, A592D, A592G, A592R, A592T, P593A, P593T, Q594T, Q594A, Q594I, Q594S, Q594D, I595A, I595T, I595V, I595T, I595S, I595Y, G596Q, G596S, G596A, G596E, T597A, T597L, T597D, T597S, T597N, T597V, T597W, T597M, V598D.

In one embodiment, the capsid protein comprise a substituted amino acid sequence at the amino acids corresponding to amino acid position 585 to 597 or 585 to 598 of the native AAV 8 (SEQ ID NO:1).

In one embodiment, the capsid protein comprise a substituted amino acid sequence of Formula I at the amino acids corresponding to amino acid position 585 to 598 of the native AAV 8 (SEQ ID NO:1): X₁X₂X₃X₄X₅X₆X₇X₈X₉X₁₀X₁₁X₁₂X₁₃X₁₄, wherein

X₁ is selected from Asn and Tyr,
X₂ is selected from Leu, Asn, Gln, Lys, His, and Phe,
X₃ is selected RECTIFIED SHEET (RULE 91),
X₄ is selected from Gln, Asn, Ser, Ala, Asp, and Gly,
X₅ is selected from Gln, Thr, Ala, Gly, Ser, and Asn,
X₆ is selected from Asn, Ala, Ser, Asp, Thr, and Gln,
X₇ is selected from Thr, Ser, Ala, Arg, Glu, and Gly,
X₈ is selected from Ala, Gln, Asp, Gly, Arg, and Thr,
X₉ is selected from Pro, Ala, and Thr,
X₁₀ is selected from Gln, Thr, Ala, Ile, Ser, and Asp,
X₁₁ is selected from Ile, Ala, Thr, Val, Thr, Ser, and Tyr
X₁₂ is selected from Gly, Gln, Ser, Ala, and Glu,
X₁₃ is selected from Thr, Ala, Leu, Asp, Ser, Asn, Val, Trp, and Met,
X₁₄ is selected from Val and Asp,
the sequence doesn't comprise an amino acids sequence of SEQ ID NO:2 (native AAV8 VR VIII).

In one embodiment, the capsid protein comprise a substituted amino acid sequence of Formula IV at the amino acids corresponding to amino acid position 585 to 597 of the native AAV 8 (SEQ ID NO:1): X₁X₂X₃X₄X₅X₆X₇X₈X₉X₁₀X₁₁X₁₂X₁₃, wherein

### X₁ is Asn,

X₂ is selected from Leu, Asn, His, and Phe,

### X₃ is Gln,

X₄ is selected from Gln, Asn, Ser, and Ala,
X₅ is selected from Gln, Thr, Ala, Gly, Ser, and Asn,
X₆ is selected from Asn, Thr, and Gln,
X₇ is selected from Thr, Ser, and Ala,
X₈ is selected from Ala, Gln, Gly, and Arg,
X₉ is selected from Pro and Ala,
X₁₀ is selected from Gln, Thr, Ala, Ile, Ser, and Asp,
X₁₁ is selected from Ile, Ala, Thr, and Val
X₁₂ is selected from Gly, Gln, Ser, Ala, and Glu,
X₁₃ is selected from Thr, Ala, Leu, Asp, Asn, Val, Trp, and Met,
the sequence doesn't comprise a amino acids sequence of SEQ ID NO:2 (native AAV8 VR VIII).

In one embodiment, the capsid protein comprise a substituted amino acid sequence of Formula II at the amino acids corresponding to amino acid position 585 to 597 of the native AAV 8 (SEQ ID NO:1): X₁X₂X₃X₄X₅X₆X₇X₈X₉X₁₀X₁₁X₁₂X₁₃, wherein

### X₁ is Asn,

X₂ is selected from Leu, Asn, and Phe,

### X₃ is Gln,

X₄ is selected from Gln, Asn, Ser, and Ala,
X₅ is selected from Thr, Ala, and Ser,
X₆ is selected from Asn, Ser, and Thr,
X₇ is selected from Thr, Ala, and Gly,
X₈ is selected from Ala, Gln, Gly, and Arg,
X₉ is selected from Pro and Ala,
X₁₀ is selected from Gln, Ala, and Ile,
X₁₁ is selected from Thr and Val,
X₁₂ is selected from Gly and Gln,
X₁₃ is selected from Thr, Leu, Asn, and Asp.

In the invention, the NCBI Reference Sequence of WT AAV8 capsid protein is YP_077180.1 (GenBank: AAN03857.1), as shown in SEQ ID NO:1.

(Amino Acid Sequence of WT AAV8 capsid)
SEQ ID NO: 1
MAADGYLPDWLEDNLSEGIREWWALKPGAPKPKANQQKQDDGRGLVLPGYKYLGPFNGLDKGEPVNA
ADAAALEHDKAYDQQLQAGDNPYLRYNHADAEFQERLQEDTSFGGNLGRAVFQAKKRVLEPLGLVEEG
AKTAPGKKRPVEPSPQRSPDSSTGIGKKGQQPARKRLNFGQTGDSESVPDPQPLGEPPAAPSGVGPNTMAA
GGGAPMADNNEGADGVGSSSGNWHCDSTWLGDRVITTSTRTWALPTYNNHLYKQISNGTSGGATNDNT
YFGYSTPWGYFDFNRFHCHFSPRDWQRLINNNWGFRPKRLSFKLFNIQVKEVTQNEGTKTIANNLTSTIQV
FTDSEYQLPYVLGSAHQGCLPPFPADVFMIPQYGYLTLNNGSQAVGRSSFYCLEYFPSQMLRTGNNFQFTY
TFEDVPFHSSYAHSQSLDRLMNPLIDQYLYYLSRTQTTGGTANTQTLGFSQGGPNTMANQAKNWLPGPCY
RQQRVSTTTGQNNNSNFAWTAGTKYHLNGRNSLANPGIAMATHKDDEERFFPSNGILIFGKQNAARDNA
DYSDVMLTSEEEIKTTNPVATEEYGIVADNLQQQNTAPQIGTVNSQGALPGMVWQNRDVYLQGPIWAKIP
HTDGNFHPSPLMGGFGLKHPPPQILIKNTPVPADPPTTFNQSKLNSFITQYSTGQVSVEIEWELQKENSKRW
NPEIQYTSNYYKSTSVDFAVNTEGVYSEPRPIGTRYLTRNL*
The DNA sequence of WT AAV8 capsid is
atggctgccgatggttatcttccagattggctcgaggacaacctctctgagggcattcgcgagtggtgggcgctgaaacctggagccccgaagccca
aagccaaccagcaaaagcaggacgacggccggggtctggtgcttcctggctacaagtacctcggacccttcaacggactcgacaagggggagcccgt
caacgcggcggacgcagcggccctggagcacgacaaggcctacgaccagcagctgcaggcgggtgacaatccgtacctgcggtataaccacgccgac
gccgagtttcaggagcgtctgcaagaagatacgtcttttgggggcaacctcgggcgagcagtcttccaggccaagaagcgggttctcgaacctctcg
gtctggttgaggaaggcgctaagacggctcctggaaagaagagaccggtagagccatcaccccagcgttctccagactcctctacgggcatcggcaa
gaaaggccaacagcccgccagaaaaagactcaattttggtcagactggcgactcagagtcagttccagaccctcaacctctcggagaacctccagca
gcgccctctggtgtgggacctaatacaatggctgcaggcggtggcgcaccaatggcagacaataacgaaggcgccgacggagtgggtagttcctcgg
gaaattggcattgcgattccacatggctgggcgacagagtcatcaccaccagcacccgaacctgggccctgcccacctacaacaaccacctctacaa
gcaaatctccaacgggacatcgggaggagccaccaacgacaacacctacttcggctacagcaccccctgggggtattttgactttaacagattccac
tgccacttttcaccacgtgactggcagcgactcatcaacaacaactggggattccggcccaagagactcagcttcaagctcttcaacatccaggtca
aggaggtcacgcagaatgaaggcaccaagaccatcgccaataacctcaccagcaccatccaggtgtttacggactcggagtaccagctgccgtacgt
tctcggctctgcccaccagggctgcctgcctccgttcccggcggacgtgttcatgattccccagtacggctacctaacactcaacaacggtagtcag
gccgtgggacgctcctccttctactgcctggaatactttccttcgcagatgctgagaaccggcaacaacttccagtttacttacaccttcgaggacg
tgcctttccacagcagctacgcccacagccagagcttggaccggctgatgaatcctctgattgaccagtacctgtactacttgtctcggactcaaac
aacaggaggcacggcaaatacgcagactctgggcttcagccaaggtgggcctaatacaatggccaatcaggcaaagaactggctgccaggaccctgt
taccgccaacaacgcgtctcaacgacaaccgggcaaaacaacaatagcaactttgcctggactgctgggaccaaataccatctgaatggaagaaatt
cattggctaatcctggcatcgctatggcaacacacaaagacgacgaggagcgtttttttcccagtaacgggatcctgatttttggcaaacaaaatgc
tgccagagacaatgcggattacagcgatgtcatgctcaccagcgaggaagaaatcaaaaccactaaccctgtggctacagaggaatacggtatcgtg
gcagataacttgcagcagcaaaacacggctcctcaaattggaactgtcaacagccagggggccttacccggtatggtctggcagaaccgggacgtgt
acctgcagggtcccatctgggccaagattcctcacacggacggcaacttccacccgtctccgctgatgggcggctttggcctgaaacatcctccgcc
tcagatcctgatcaagaacacgcctgtacctgcggatcctccgaccaccttcaaccagtcaaagctgaactctttcatcacgcaatacagcaccgga
caggtcagcgtggaaattgaatgggagctgcagaaggaaaacagcaagcgctggaaccccgagatccagtacacctccaactactacaaatctacaa
gtgtggactttgctgttaatacagaaggcgtgtactctgaaccccgccccattggcacccgttacctcacccgtaatctgtaa

In one specific embodiment, the present invention provides a variant AAV 8 capsid protein, comprising a substituted sequence corresponding to the position amino acids 585 to 597 of SEQ ID NO:1 (AAV8); preferably, the sequence comprises a amino acids sequence selected from the groups consisting of SEQ ID NO:3-42 as shown in Table 6, preferably selected from the groups consisting of SEQ ID NO: 2-3, 6-7, 9-11, 13-14, 16, 20-22, 24, 25, 32-33, 37, 39, 42 as shown in Table 10.

In one specific embodiment, the present invention provides a variant AAV 8 capsid protein, comprising a substituted sequence corresponding to the position amino acids 585 to 597 of SEQ ID NO:1 (AAV8); preferably, the sequence comprises a amino acids sequence selected from the groups consisting of SEQ ID NO:21 (AAV 8-Lib20), SEQ ID NO:25 (AAV 8-Lib25), SEQ ID NO:9 (AAV 8-Lib43), and SEQ ID NO:37 (AAV 8-Lib44).

In some embodiment, the AAV variant is AAV serotype 9. The present invention provides an AAV library comprising a multitude of adeno-associated virus (AAV) variants, the AAV variants comprises a variant AAV capsid protein comprising a substitution at one or more of amino acid residues N583, H584, Q585, 5586, A587, Q588, A589, Q590, A591, Q592, T593, G594, W595, V596, corresponding to amino acid sequence of the native AAV 9 (SEQ ID NO:43), the substitution of amino acid residues is selected from N583Y, H584N, H584Q, H584K, H584L, H584F, Q585N, S586N, S586Q, S586A, S586D, S586G, A587T, A587Q, A587G, A587S, A587N, Q588A, Q588S, Q588D, Q588T, Q588N, A589S, A 589T, A589R, A589E, A589G, Q590A, Q590D, Q590G, Q590R, Q590T, A591P, A591T, Q592T, Q592A, Q5921, Q592S, Q592D, T593A, T5931, T593V, T593S, T593Y, G594Q, G594S, G594A, G594E, W595A, W595L, W595D, W595S, W595N, W595V, W 595T, W595M, V596D.

In one embodiment, the present invention provides a variant AAV 9 capsid protein a substituted amino acid sequence corresponding to VR VIII region of the native AAV9 capsid protein. The capsid protein comprise a substituted amino acid sequence of Formula I at the amino acids corresponding to amino acid position 583 to 596 of the native AAV 9 (SEQ ID NO:43): X₁X₂X₃X₄X₅X₆X₇X₈X₉X₁₀X₁₁X₁₂X₁₃X₁₄, wherein

X₁ is selected from Asn and Tyr,
X₂ is selected from Leu, Asn, Gln, Lys, His, and Phe,
X₃ is selected from Gln and Asn,
X₄ is selected from Gln, Asn, Ser, Ala, Asp, and Gly,
X₅ is selected from Gln, Thr, Ala, Gly, Ser, and Asn,
X₆ is selected from Asn, Ala, Ser, Asp, Thr, and Gln,
X₇ is selected from Thr, Ser, Ala, Arg, Glu, and Gly,
X₈ is selected from Ala, Gln, Asp, Gly, Arg, and Thr,
X₉ is selected from Pro, Ala, and Thr,
X₁₀ is selected from Gln, Thr, Ala, Ile, Ser, and Asp,
X₁₁ is selected RECTIFIED SHEET (RULE91); Val, Thr, Ser, and Tyr
X₁₂ is selected from Gly, Gln, Ser, Ala, and Glu,
X₁₃ is selected from Thr, Ala, Leu, Asp, Ser, Asn, Val, Trp, and Met,
X₁₄ is selected from Val, and Asp,
the sequence doesn't comprise a amino acids sequence of SEQ ID NO:33 (native AAV9 VR VIII).

In one embodiment, VR VIII region is the position amino acids 583 to 595 of SEQ ID NO:43 (AAV9), as compared to a wild-type AAV9 capsid proteins; the capsid protein comprise a substituted amino acid sequence of Formula II at the amino acids corresponding to amino acid position 585 to 597 of the native AAV 9 (SEQ ID NO:43): X₁X₂X₃X₄X₅X₆X₇X₈X₉X₁₀X₁₁X₁₂X₁₃, wherein

### X₁ is Asn,

### X₂ is Leu,

### X₃ is Gln,

### X₄ is Asn, or Ser,

X₅ is selected from Ala, Ser, and Gly,

### X₆ is Asn,

### X₇ is Thr

X₈ is selected from Ala, Gln, and Gly,

### X₉ is Pro, or Ala,

X₁₀ is selected from Gln, Thr, and Ala,

### X₁₁ is Thr,

X₁₂ is selected from Gly, Gln, Ala, and Glu,
X₁₃ is selected from Thr, Asn, and Asp.

In the invention, the NCBI Reference Sequence of WT AAV9 capsid protein is AAS99264.1 (GenBank: ANF53541.1), as shown in SEQ ID NO:43.

(Amino Acid Sequence of WT A AV9 capsid)
SEQ ID NO: 43
MAADGYLPDWLEDNLSEGIREWWALKPGAPQPKANQQHQDNARGLVLPGYKYLGPGNGLDKGEPVNA
ADAAALEHDKAYDQQLKAGDNPYLKYNHADAEFQERLKEDTSFGGNLGRAVFQAKKRLLEPLGLVEEA
AKTAPGKKRPVEQSPQEPDSSAGIGKSGAQPAKKRLNFGQTGDTESVPDPQPIGEPPAAPSGVGSLTMASG
GGAPVADNNEGADGVGSSSGNWHCDSQWLGDRVITTSTRTWALPTYNNHLYKQISNSTSGGSSNDNAYF
GYSTPWGYFDFNRFHCHFSPRDWQRLINNNWGFRPKRLNFKLFNIQVKEVTDNNGVKTIANNLTSTVQV
FTDSDYQLPYVLGSAHEGCLPPFPADVFMIPQYGYLTLNDGSQAVGRSSFYCLEYFPSQMLRTGNNFQFSY
EFENVPFHSSYAHSQSLDRLMNPLIDQYLYYLSKTINGSGQNQQTLKFSVAGPSNMAVQGRNYIPGPSYRQ
QRVSTTVTQNNNSEFAWPGASSWALNGRNSLMNPGPAMASHKEGEDRFFPLSGSLIFGKQGTGRDNVDA
DKVMITNEEEIKTTNPVATESYGQVATNHQSAQAQAQTGWVQNQGILPGMVWQDRDVYLQGPIWAKIPH
TDGNFHPSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL
The DNA sequence of WT AAV9 capsid is
atggctgccgatggttatcttccagattggctcgaggacaaccttagtgaaggaattcgcgagtggtgggctttgaaacctggagcccctcaaccca
aggcaaatcaacaacatcaagacaacgctcgaggtcttgtgcttccgggttacaaataccttggacccggcaacggactcgacaagggggagccggt
caacgcagcagacgcggcggccctcgagcacgacaaggcctacgaccagcagctcaaggccggagacaacccgtacctcaagtacaaccacgccgac
gccgagttccaggagcggctcaaagaagatacgtcttttgggggcaacctcgggcgagcagtcttccaggccaaaaagaggcttcttgaacctcttg
gtctggttgaggaagcggctaagacggctcctggaaagaagaggcctgtagagcagtctcctcaggaaccggactcctccgcgggtattggcaaatc
gggtgcacagcccgctaaaaagagactcaatttcggtcagactggcgacacagagtcagtcccagaccctcaaccaatcggagaacctcccgcagcc
ccctcaggtgtgggatctcttacaatggcttcaggtggtggcgcaccagtggcagacaataacgaaggtgccgatggagtgggtagttcctcgggaa
attggcattgcgattcccaatggctgggggacagagtcatcaccaccagcacccgaacctgggccctgcccacctacaacaatcacctctacaagca
aatctccaacagcacatctggaggatcttcaaatgacaacgcctacttcggctacagcaccccctgggggtattttgacttcaacagattccactgc
cacttctcaccacgtgactggcagcgactcatcaacaacaactggggattccggcctaagcgactcaacttcaagctcttcaacattcaggtcaaag
aggttacggacaacaatggagtcaagaccatcgccaataaccttaccagcacggtccaggtcttcacggactcagactatcagctcccgtacgtgct
cgggtcggctcacgagggctgcctcccgccgttcccagcggacgttttcatgattcctcagtacgggtatctgacgcttaatgatggaagccaggcc
gtgggtcgttcgtccttttactgcctggaatatttcccgtcgcaaatgctaagaacgggtaacaacttccagttcagctacgagtttgagaacgtac
ctttccatagcagctacgctcacagccaaagcctggaccgactaatgaatccactcatcgaccaatacttgtactatctctcaaagactattaacgg
ttctggacagaatcaacaaacgctaaaattcagtgtggccggacccagcaacatggctgtccagggaagaaactacatacctggacccagctaccga
caacaacgtgtctcaaccactgtgactcaaaacaacaacagcgaatttgcttggcctggagcttcttcttgggctctcaatggacgtaatagcttga
tgaatcctggacctgctatggccagccacaaagaaggagaggaccgtttctttcctttgtctggatctttaatttttggcaaacaaggaactggaag
agacaacgtggatgcggacaaagtcatgataaccaacgaagaagaaattaaaactactaacccggtagcaacggagtcctatggacaagtggccaca
aaccaccagagtgcccaagcacaggcgcagaccggctgggttcaaaaccaaggaatacttccgggtatggtttggcaggacagagatgtgtacctgc
aaggacccatttgggccaaaattcctcacacggacggcaactttcacccttctccgctgatgggagggtttggaatgaagcacccgcctcctcagat
cctcatcaaaaacacacctgtacctgcggatcctccaacggccttcaacaaggacaagctgaactctttcatcacccagtattctactggccaagtc
agcgtggagatcgagtgggagctgcagaaggaaaacagcaagcgctggaacccggagatccagtacacttccaactattacaagtctaataatgttg
aatttgctgttaatactgaaggtgtatatagtgaaccccgccccattggcaccagatacctgactcgtaatctgtaa

In one specific embodiment, the present invention provides a variant AAV 9 capsid protein, comprising a substituted sequence corresponding to the position amino acids 583 to 595 or 596 of SEQ ID NO:43 (AAV9); preferably, the sequence comprises a amino acids sequence selected from the groups consisting of SEQ ID NO: 3-42 as shown in Table 8.

In one specific embodiment, the present invention provides a variant AAV 9 capsid protein, comprising a substituted sequence corresponding to the position amino acids 583 to 595 of SEQ ID NO:43 (AAV9); preferably, the sequence comprises a amino acids sequence selected from the groups consisting of SEQ ID NO:29 (AAV 9-Lib31), SEQ ID NO:14 (AAV 9-Lib 33), SEQ ID NO:9 (AAV 9-Lib43), and SEQ ID NO:11 (AAV 9-Lib46).

In another aspect, the present invention provides an isolated polynucleotide comprising a nucleotide sequence that encodes above variant AAV capsid protein or vectors comprising the above polynucleotides.

The present invention provides an isolated, genetically modified host cell comprising the above polynucleotide.

The present invention provides a recombinant AAV virion comprising above variant AAV capsid protein.

In one specific embodiment, the present invention provides a pharmaceutical composition comprising:

a) a recombinant adeno-associated virus virion disclosed in the present invention; and
b) a pharmaceutically acceptable excipient.

In another aspect, the present invention provides a recombinant AAV vector, comprising polynucleotide encoding a variant AAV capsid protein of invention, and an AAV 5′ inverted terminal repeat (ITR), an engineered nucleic acid sequence encoding a functional gene product, a regulatory sequence which directs expression of the gene product in a target cell, and an AAV 3′ ITR.

In one specific embodiment, the regulatory sequence further comprises at least of an enhancer, a promoter, intron, and poly A.

In another aspect, the present invention also provides a method of delivering a nucleic acid vector encoding a functional gene product to cells and/or tissues with a recombinant AAV virion or recombinant AAV vector of the present invention.

In one specific embodiment, the present invention also provides the use of a recombinant AAV virion or recombinant AAV vector of the present invention in the preparation of product for delivering a nucleic acid vector encoding a functional gene product to cells and/or tissues.

In another aspect, the present invention also provides a method of treating disease, the method comprising administering to a subject in need thereof an effective amount of a recombinant AAV virion of present invention, the recombinant AAV virion comprises functional gene product.

In one specific embodiment, the present invention also provides the use of a recombinant AAV virion or recombinant AAV vector of the present invention in the preparation of product for treating disease to a subject in need thereof; preferably, the disease is selected from the group consisting of liver disease, central nervous system diseases, and other diseases.

In one specific embodiment, the gene product is a polypeptide.

In one specific embodiment, the polypeptide is selected from the group consisting of Cystathionine-beta-synthase (CBS), Factor IX (FIX), Factor VIII (F8), Glucose-6-phosphatase catalytic subunit (G6PC), Glucose 6-phosphatase (G6Pase), Glucuronidase, beta (GUSB), Hemochromatosis (HFE), Iduronate 2-sulfatase (IDS), Iduronidase, alpha-1 (IDUA), Low density lipoprotein receptor (LDLR), Myophosphorylase (PYGM), N-acetylglucosaminidase, alpha (NAGLU), N-sulfoglucosamine sulfohydrolase (SGSH), Ornithine carbamoyltransferase (OTC), Phenylalanine hydroxylase (PAH), UDP glucuronosyltransferase 1 family, polypeptide A1 (UGT1A1); preferably wherein the recombinant AAV virion comprises a variant AAV8 capsid protein comprising a amino acids sequence selected from the groups consisting of SEQ ID NO: 2-3, 6-7, 9-11, 13-14, 16, 20-22, 24, 25, 32-33, 37, 39, 42 as shown in Table 10, preferably SEQ ID NO:21 (AAV8-Lib20), SEQ ID NO:25 (AAV8-Lib25), SEQ ID NO:9 (AAV8-Lib43), and SEQ ID NO:37 (AAV8-Lib44), and comprises a variant AAV9 capsid protein comprising a amino acids sequence of SEQ ID NO:11 (AAV9-Lib46).

In one specific embodiment, the polypeptide is selected from the group consisting of Acid alpha-glucosidase (GAA), ApaL1, Aromatic L-amino acid decarboxylase (AADC), Aspartoacylase (ASPA), Battenin, Ceroid lipofuscinosis neuronal 2 (CLN2), Cluster of Differentiation 86 (CD86 or B7-2), Cystathionine-beta-synthase (CBS), Dystrophin or Mini dystrophin, Frataxin (FXN), Glial cell-derived neurotrophic factor (GDNF), Glutamate decarboxylase 1 (GAD1), Glutamate decarboxylase 2 (GAD2), Hexosaminidase A, α polypeptide, also called beta-Hexosaminidase alpha (HEXA), Hexosaminidase B, β polypeptide, also called beta-Hexosaminidase beta (HEXB), Interleukin 12 (IL-12), Methyl CpG binding protein 2 (MECP2), Myotubularin 1 (MTM1), NADH ubiquinone oxidoreductase subunit 4 (ND4), Nerve growth factor (NGF), neuropeptide Y (NPY), Neurturin (NRTN), Palmitoyl-protein thioesterase 1 (PPT1), Sarcoglycan alpha, beta, gamma, delta, epsilon, or zeta (SGCA, SGCB, SGCG, SGCD, SGCE, or SGCZ), Tumor necrosis factor receptor fused to an antibody Fc (TNFR:Fc), Ubiquitin-protein ligase E3A (UBE3A), β-galactosidase 1 (GLB1); preferably wherein the recombinant AAV virion comprises a variant AAV9 capsid protein comprising a amino acids sequence selected from the groups consisting of SEQ ID NOs:9 (AAV9-Lib43) and SEQ ID NOs:11 (AAV9-Lib46).

In one specific embodiment, the polypeptide is selected from the group consisting of Adenine nucleotide translocator (ANT-1), Alpha-1-antitrypsin (AAT), Aquaporin 1 (AQP1), ATPase copper transporting alpha (ATP7A), ATPase, Ca++ transporting, cardiac muscle, slow twitch 2 (SERCA2), C1 esterase inhibitor (ClEI), Cyclic nucleotide gated channel alpha 3 (CNGA3), Cyclic nucleotide gated channel beta 3 (CNGB3), Cystic fibrosis transmembrane conductance regulator (CFTR), Galactosidase, alpha (AGA), Glucocerebrosidase (GC), Granulocyte-macrophage colonystimulating factory (GM-CSF), HIV-1 gag-proArt (tgAAC09), Lipoprotein lipase (LPL), Medium-chain acyl-CoA dehydrogenase (MCAD), Myosin 7A (MYO7A), Poly(A) binding protein nuclear 1 (PABPN1), Propionyl CoA carboxylase, alpha polypeptide (PCCA), Rab escort protein-1 (REP-1), Retinal pigment epithelium-specific protein 65 kDa (RPE65), Retinoschisin 1 (RS1), Short-chain acyl-CoA dehydrogenase (SCAD), Very long-acyl-CoA dehydrogenase (VLCAD).

### BRIEF DESCRIPTION OF THE DRAWINGS

FIG. 1 shows the outline of in vivo screen strategy.

FIG. 2 shows the screen results. A) The week 1 screen result for liver. B) The week 1 screen result for brain. C) The week 4 screen result for various tissues. The result of starting library was marked in blue line.

FIG. 3 shows the effect of AAV8-VR VIII variants. A) Luciferase expression in HEK293T cells transduced with AAV8 and AAV8-VR VIII variants. MOI=10,000, n=3. B) In vivo luciferase expression in C57BL/6J mice 3 days after 1×10\hat{ }10 vg of control AAV8 and AAV8-VR VIII variants following intravenous injection. Negative control, PBS injected animals. The same results were observed in two indepenRECTIFIED SHEET(RULE91) peats. C) Luciferase quantification of AAV8 and AAV8-VR VIII variants in C57BL/6J animals or PBS control at day 3, 7 and 14. n=6 Data are reported as mean±SEM. D) Luciferase quantification of AAV8 and AAV8-VR VIII variants in C57BL/6J animals or PBS control at day 3. E) Luciferase quantification of AAV8 and AAV8-VR VIII variants in C57BL/6J animals or PBS control at day 7. F) Luciferase quantification of AAV8 and AAV8-VR VIII variants in C57BL/6J animals or PBS control at day 14. The same results were observed in two independent biological repeats.

FIG. 4 shows at week 2 post injection, lung, liver, spleen, heart, kidney, lymph node, right quadriceps (LQ), left quadriceps (LQ) muscle and brain were harvested to detect the vector genome copy number in each tissue, n=6. The absolute GCNs in different tissues were plotted together for AAV8 (A), AAV8-Lib20 (B), AAV8-Lib25 (C), AAV8-Lib43 (D), AAV8-Lib44 (E), AAV8-Lib45 (F). The same results were observed in two independent biological repeats.

FIG. 5 shows the liver GCNs among different AAV8 VR VIII variants. Data are reported as mean±SEM.

FIG. 6 shows at week 2 post injection, we determined the serum alanine transaminase (ALT) level. No significant change was noticed between control (PBS and AAV8) and AAV8-VR VIII variants.

FIG. 7 shows the effect of AAV9-VR VIII variants. A) In vivo luciferase expression in C57BL/6J mice 7 days after 1×10\hat{ }11 vg of control AAV9 and AAV9-VR VIII variants following intravenous injection. Negative control, PBS injected animals. B) Luciferase quantification of AAV9 and AAV9-VR VIII variants in C57BL/6J animals or PBS control. Data are reported as mean±SEM. C) Luciferase expression and quantification (D) of AAV9 and AAV9-VR VIII variants in the head of C57BL/6J animals or PBS control. n=6 Data are reported as mean±SEM. E) The head/body ratio of AAV9 and AAV9-VR VIII variants. For all the above experiments, the same results were observed in two independent biological repeats.

FIG. 8 shows luciferase expression in HEK293T cells transduced with AAV9 and AAV9-VR VIII variants. MOI=10,000, n=3.

FIG. 9 shows at week 2 post injection, tissues were harvested to detect the vector genome copy number, n=6. The absolute GCNs in each tissues were plotted for liver (A), brain (B), heart (C), and Lung (D). The same results were observed in two independent biological repeats.

FIG. 10 shows ALT level following AAV9 VR VIII variants—mediated gene delivery.

FIG. 11 shows the effect of AAV2-VR VIII variants. A) Luciferase expression in HEK293T cells transduced with AAV2 and AAV2-VR VIII variants. MOI=10,000, n=3. B) In vivo luciferase expression in C57BL/6J mice 3 days after 1×10\hat{ }10 vg of control AAV2 and AAV2-VR VIII variants following intravenous injection. Negative control, PBS injected animals. The same results were observed in two independent biological repeats. C) Luciferase quantification of AAV2 and AAV2-VR VIII variants in C57BL/6J animals or PBS control at day 3, 7 and 14. n=6 Data are reported as mean±SEM.

FIG. 12 shows the effect of AAV2-VR VIII variants. A) In vivo luciferase expression in C57BL/6J mice 7 days after 1×10\hat{ }10 vg of control AAV2 and AAV2-VR VIII variants following intravenous injection. Negative control, PBS injected animals. B) Luciferase quantification of AAV2 and AAV8-VR VIII variants in C57BL/6J animals or PBS control at day 7. C) In vivo luciferase expression in C57BL/6J mice 14 days after 1×10\hat{ }10 vg of control AAV2 and AAV2-VR VIII variants following intravenous injection. Negative control, PBS injected animals. D) Luciferase quantification of AAV8 and AAV8-VR VIII variants in C57BL/6J animals or PBS control at day 14. The same results were observed in two independent biological repeats.

FIG. 13 shows hFIX expression in monkey plasma.

### DETAILED DESCRIPTION

The following description of the disclosure is merely intended to illustrate various embodiments of the disclosure. As such, the specific modifications discussed are not to be construed as limitations on the scope of the disclosure. It will be apparent to one skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety.

The articles “a”, “an”, and “the” are used herein to refer to one or to more than one (i.e., to at least one) of the grammatical object of the article. By way of example, “a polypeptide complex” means one polypeptide complex or more than one polypeptide complex.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%.

Throughout this disclosure, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

### Pharmaceutical Composition

The present disclosure also provides a pharmaceutical composition comprising the polypeptide complex or the bispecific polypeptide complex provided herein and a pharmaceutically acceptable carrier.

The term “pharmaceutically acceptable” indicates that the designated carrier, vehicle, diluent, excipient (s), and/or salt is generally chemically and/or physically compatible with the other ingredients comprising the formulation, and physiologically compatible with the recipient thereof.

A “pharmaceutically acceptable carrier” refers to an ingredient in a pharmaceutical formulation, other than an active ingredient, which is bioactivity acceptable and nontoxic to a subject. Pharmaceutical acceptable carriers for use in the pharmaceutical compositions disclosed herein may include, for example, pharmaceutically acceptable liquid, gel, or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

### Method of Treatment

Therapeutic methods are also provided, comprising: administering a therapeutically effective amount of the polypeptide complex or the bispecific polypeptide complex provided herein to a subject in need thereof, thereby treating or preventing a condition or a disorder. In certain embodiments, the subject has been identified as having a disorder or condition likely to respond to the polypeptide complex or the bispecific polypeptide complex provided herein.

As used herein, the term “subject” includes any human or nonhuman animal. The term “nonhuman animal” includes all vertebrates, e.g., mammals and non-mammals, such as nonhuman primates, sheep, dogs, cats, horses, cows, chickens, amphibians, reptiles, etc. Except when noted, the terms “patient” or “subject” are used interchangeably.

The terms “treatment” and “therapeutic method” refer to both therapeutic treatment and prophylactic/preventative measures. Those in need of treatment may include individuals already having a particular medical disorder as well as those who may ultimately acquire the disorder.

In certain embodiments, the conditions and disorders include tumors and cancers, for example, non-small cell lung cancer, small cell lung cancer, renal cell cancer, colorectal cancer, ovarian cancer, breast cancer, pancreatic cancer, gastric carcinoma, bladder cancer, esophageal cancer, mesothelioma, melanoma, head and neck cancer, thyroid cancer, sarcoma, prostate cancer, glioblastoma, cervical cancer, thymic carcinoma, leukemia, lymphomas, myelomas, mycoses fungoids, merkel cell cancer, and other hematologic malignancies, such as classical Hodgkin lymphoma (CHL), primary mediastinal large B-cell lymphoma, T-cell/histiocyte-rich B-cell lymphoma, EBV-positive and -negative PTLD, and EBV-associated diffuse large B-cell lymphoma (DLBCL), plasmablastic lymphoma, extranodal NK/T-cell lymphoma, nasopharyngeal carcinoma, and HHV8-associated primary effusion lymphoma, Hodgkin's lymphoma, neoplasm of the central nervous system (CNS), such as primary CNS lymphoma, spinal axis tumor, brain stem glioma.

### EXAMPLES

### Example 1: The Equipments and Regents

TABLE 1
The equipment used in the invention

| Equipments | Product number | Supplier |
|---|---|---|
| SpectraMax ® M5/M5e Multimode | SpectraMax ® M5/M5e | Molecular Devices |
| Plate Reader | | |
| Diagnostica Stago STart 4 | ST art | Diagnostica Stago |
| Hemostasis Analyzer | | |
| EnVision 2105 multimode plate | 2105-0010 | PerkinElmer |
| reader | | |
| Ice machine | ST150 | Sciencetool |
| CYRO Vessel Locator 4 PLUS | CY50935-70 | Thermo Fisher Scientific |
| 4° C. refrigerator | HYC390F | Haier |
| −20° C. refrigerator | DW-40L348 | Haier |
| −80° C. refrigerator | 8960086V | Thermo Fisher Scientific |
| Biosafety cabinet | BSC-II-A2 | Sujing |
| Incubator | HERAcell 240i | Thermo Fisher Scientific |
| Countess ™ II cell counter | AMQAX1000 | Thermo Fisher Scientific |
| Inverted Microscope | ECLIPS TS2 | Nikon |
| Refrigerated centrifuge | 5424R | Eppendorf |
| Centrifuge | 5810R | Eppendorf |
| Ultracentrifuge | Optima XPN-100 | Beckman Coulter |
| Basic Power Supply | PowerPac Basic | Bio-Rad |
| Amersham Imager 680 blot and | Amersham Imager 680QC | GE Healthcare |
| gel imager | | |
| NanoDrop One Microvolume | NanoDrop One/One^{c} UV-Vis | Invitrogen |
| UV-Vis Spectrophotometers | | |
| Applied Biosystems | QuantStudio ™ 7 | Applied Biosystems |
| QuantStudio ™ 7 Flex Real-Time | | |
| PCR System | | |
| ProFlex ™ 3 × 32-well PCR | ProFlex ™ 3 × 32-well PCR | Thermo Fisher Scientific |
| System | System-4484073 | |
| Tanon 2500/2500R Gel Imaging | 2500R | Tanon |
| System | | |
| Milli-Q ® Direct 8 Water | Direct 8 | EMD Millipore |
| Purification System | | |

TABLE 2
The regents and supplies used in the study

| | Reagents & Supplies | |
|---|---|---|
| Cell culture | DMEM, High Glucose | Gibco 11965118 |
| | HEK293T | ATCC CRL-3216 ™ |
| | Trypsin-EDTA (0.25%), phenol red | Invitrogen 25200072 |
| | DPBS | Corning 21-031-CV |
| | FBS | Corning 35-081-CV |
| | DMSO | Sigma-Aldrich D2650 |
| | Antibiotic-Antimycotic, 100X | Gibco 15240062 |
| | Countess ™ Cell Counting Chamber Slides | Invitrogen C10312 |
| | Corning ® 150 mm TC-treated Culture | Corning 430599 |
| | Dish | |
| | 1.5 mL MaxyClear Snaplock | Axygen Mct-150-C |
| | Microcentrifuge Tube | |
| Construction of AAV | NdeI | NEB R0111S |
| plasmid | XbaI | NEB R0145S |
| | NEBuilder HiFi DNA Assembly Master | NEB E2621L |
| | Mix | |
| | Ampicillin Sodium(100 mg/ml) | TIANGEN RT501 |
| | Endura Competent Cells | Lucigen 60241-2 |
| | 0.2 mL Polypropylene PCR Tube Strips, 8 | Axygen PCR-0208-C |
| | Tubes/Strip | |
| | 8-Strip PCR Tube Caps for 0.2 mL PCR | Axygen PCR-02CP-C |
| | Tube Strips, Clear PP | |
| AAV VR VIII variants | PEI 25K | Polysciences 23966-1 |
| packaging, mixing for in | EndoFree Plasmid Maxi Kit | QIAGEN 12362 |
| vivo selection & | Benzonase | Novagen 70664 |
| Recombinant AAV | OptiPrep ™ Density Gradient Medium | Sigma-Aldrich D1556 |
| packaging | Power SYBR ™ Green PCR Master Mix | Applied Biosystems 4367659 |
| | Fisherbrand ™ Cell Lifters | Fisher Scientific 08100240 |
| | Quick-Seal Polypropylene Tube | Beckman Coulter 342414 |
| | APOLLO 20 mL 150 KDa Concentrators | Orbital Biosciences AP2015010 |
| | HiTrap ® Q High Performance | GE Healthcare 17-1154-01 |
| In vivo Selection for | C57BL/6J mice | Shanghai SLAC Laboratory Animal |
| liver-targeting variants | | Co. |
| | DNesay Blood&Tissue kit | QIAGEN 69506 |
| NGS to quantify the | Zymoclean ™ Gel DNA Recovery Kit | Zymo Research D4002 |
| AAV genome reads in | Agarose | Biowest 111860 |
| tissues | Marker II | TIANGEN MD102 |
| | Gel Loading Dye, Purple (6X) | NEB B7024S |
| Titration of particles by | AAV8 Titration ELISA | PROGEN- PRAAV8 |
| ELISA | Recombinant Adeno-associated virus 8 | ATCC VR-1816 |
| In vivo rAAV-luciferase | XenoLight D-Luciferin - K+ Salt | PerkinElmer 122799-10 |
| transduction and | Bioluminescent Substrate | |
| detection | ALT Activity Assay Kit | Sigma-Aldrich MAK052-1KT |
| In vivo rAAV-hFIX | F9 KO mice | Shanghai Model Oranisms |
| transduction | | |
| Tissule, plasma and | DPBS | Corning |
| serum collection | | 21-031-CV/Hyclone-SH30028.03 |
| | 3.8% sodium citrate | HIMEDIA-R014 |
| | 10% Neutral buffered formalin | INNOCHEM-A28231 |
| Detection if hFIX | VisuLize Factor IX Antigen Kit | Affinity Biologicals FIX-AG |
| expression | Rox Factor IX | Rossix 900020 |
| In vivo viral genome | Power SYBR ™ Green PCR Master Mix | Applied Biosystems 4367659 |
| copy number | DEPC-Treated water | Invitrogen AM9916 |
| | DNesay Blood&Tissue kit | QIAGEN 69506 |
| | Hard-Shell ® 384-Well PCR Plates | Bio-Rad HSP3801 |
| | Axygen ® 60 μm CyclerSeal Sealing Film | Axygen PCR-TS |
| | for Storage and PCR Application | |
| | Multiplate ™ 96-Well PCR Plates, low | Bio-Rad MLP-9601 |
| | profile | |
| In vitro Infectivity | Bright-Glo ™ Luciferase Assay System | Promega E2620 |
| | 96 Well Clear Round Bottom TC-Treated | Corning 3799 |
| | Microplate | |
| Sodium dodecyl | NuPAGE ™ 4-12% Bis-Tris Protein Gels, | Invitrogen NP0321BOX |
| sulfate-polyacrylamide | 1.0 mm, 10-well | |
| | NuPAGE ™ Sample Reducing Agent | Invitrogen NP0009 |
| | (10X) | |
| | Fast Silver Stain Kit | Beyotime P0017S |
| | BenchMark ™ Protein Ladder | Invitrogen 10747012 |

TABLE 3
The various oligos used in the study
Construction of AAV plasmids

| | | |
|---|---|---|
| pITR2-Rep2-Cap8- | Forward | 5′-TAAGCCAACTAGTGGAACCGGTGCGGCCGCACGCGTGG |
| library-ITR2-1 | | AGTTTAAGCCCGAGTGAGCACGCAGGGTCTCCATTTTGAAGCGGGAG |
| | | GTTTGAACGCGCAGCCGCCATGCCGGGGTT-3′ |
| | Reverse | 5′-GAAGATAACCATCGGCAGCCATTTAATTAAACCTGATTTAAATCATT |
| | | TATTGTTCAAAG-3′ |
| pITR2-Rep2-Cap8- | Forward | 5′-CTTTGAACAATAAATGATTTAAATCAGGTTTAATTAAATGGCTGCCG |
| library-ITR2-2 | | ATGGTTAT CTTC-3′ |
| | Reverse | 5′-TTCCAATTTGAGGAGCCGTGTTTTGCTGCTGCAA |
| | | CATATGGTTATCTGCCACGATACCGTATT-3′ |
| pITR2-Rep2-Cap8- | Forward | 5′-ACACGGCTCCTCAAATTGGAATCTAGACTGTCA |
| library-ITR2-3 | | ACAGCCAGGGGGCCTTACCCGGTATGGTCTG-3′ |
| | Reverse | 5′-GCCAACTCCATCACTAGGGGTTCCTGCGGCCGCTCGGTCCGCACG |
| | | TGGTTACCTACAAAATGCTAGCTTACAGATTACGGGTGAGGTAACG-3′ |
| Cap8-library | Forward | 5′-GCATCTTTGAACAATAAATGATTTAAATCAGGTATGGCTGCCGATG |
| region | | GTTATCT-3′ |
| | Reverse | 5′-GTTTATTGATTAACAAGCAATTACAGATTACGGGTGAGGT-3′ |
| pAAV-RC8 | Forward | 5′-TTGCTTGTTAATCAATAAACCG-3′ |
| backbone | Reverse | 5′-ACCTGATTTAAATCATTTATTGTTCAAAGATGC-3′ |
| pAAV-RC9 | Forward | 5′-TTGCTTGTTAATCAATAAACCG-3′ |
| backbone | Reverse | 5′-ACCTGATTTAAATCATTTATTGTTCAAAGATGC-3′ |

Titering and Mixing of AAV Capsid Library

| | | |
|---|---|---|
| Rep gene | Forward | 5′-GCAAGACCGGATGTTCAAAT-3′ |
| | Reverse | 5′-CCTCAACCACGTGATCCTTT-3′ |

Titering of Recombinant AAV

| | | |
|---|---|---|
| CMV promoter | Forward | 5′-TCCCATAGTAACGCCAATAGG-3′ |
| | Reverse | 5′-CTTGGCATATGATACACTTGATG-3′ |
| TTR promoter | Forward | 5′-TCCCATAGTAACGCCAATAGG-3′ |
| | Reverse | 5′-CTTGGCATATGATACACTTGATG-3′ |

Next Generation Sequencing to quantify the AAV genome reads in tissues

| | | |
|---|---|---|
| VR VIII region | Forward | 5′-CAAAATGCTGCCAGAGACAA-3′ |
| | Reverse | 5′-GTCCGTGTGAGGAATCTTGG-3′ |

In vivo viral genome copy number

| | | |
|---|---|---|
| CMV promoter | Forward | 5′-TCCCATAGTAACGCCAATAGG-3′ |
| | Reverse | 5′-CTTGGCATATGATACACTTGATG-3′ |

TABLE 4
The primers used for amplifying pAAV-RC9-library fragment1

| target | primers | sequences |
|---|---|---|
| Cap9-lib2-1 | Cap9-F | GCATCTTTGAACAATAAATGATTTAAATCAGGTATGGCTGCCGATGG |
| | | TTATCT |
| | Cap9-lib2- | AGTTTGTGTCTGGGGTGCAGTATTAGCCGATTGTAAGTTTGTGGCC |
| | R | ACTTGTCCATAGG |
| Cap9-lib7-1 | Cap9-F | GCATCTTTGAACAATAAATGATTTAAATCAGGTATGGCTGCCGATGG |
| | | TTATCT |
| | Cap9-lib7- | GGTCCCTGTTTGAGGAGCGGTGTTTGCCGATTGCAGGTTTGTGGCC |
| | R | ACTTGTCCATAGG |
| Cap9-lib31-1 | Cap9-F | GCATCTTTGAACAATAAATGATTTAAATCAGGTATGGCTGCCGATGG |
| | | TTATCT |
| | Cap9-lib31- | ATTTTCTGTAGTTGGACCAGTATTTGAGTTTTGCAAATTTGTGGCCA |
| | R | CTTGTCCATAGG |
| Cap9-lib33-1 | Cap9-F | GCATCTTTGAACAATAAATGATTTAAATCAGGTATGGCTGCCGATGG |
| | | TTATCT |
| | Cap9-lib33- | AGTTCCGGTCGCAGGGGCTGTGTTGCTGCTCTGGAGATTTGTGGC |
| | R | CACTTGTCCATAGG |
| Cap9-lib43-1 | Cap9-F | GCATCTTTGAACAATAAATGATTTAAATCAGGTATGGCTGCCGATGG |
| | | TTATCT |
| | Cap9-lib43- | GGTCCCCGTTTGAGGAGCGGTGTTTGCCGACTGTAGGTTTGTGGC |
| | R | CACTTGTCCATAGG |
| Cap9-lib11-1 | Cap9-F | GCATCTTTGAACAATAAATGATTTAAATCAGGTATGGCTGCCGATGG |
| | | TTATCT |
| | Cap9-lib11- | AGTTCCTGTAGTTGGACCAGTGTTTGAGTTTTGCAAATTTGTGGCC |
| | R | ACTTGTCCATAGG |
| Cap9-lib46-1 | Cap9-F | GCATCTTTGAACAATAAATGATTTAAATCAGGTATGGCTGCCGATGG |
| | | TTATCT |
| | Cap9-lib46- | ATCTGCGGTAGCTGCTTGTGTGTTGCCGCTCTGGAGGTTTGTGGCC |
| | R | ACTTGTCCATAGG |

TABLE 5
The primers used for amplifying pAAV-RC9-library fragment?

| target | primers | sequences |
|---|---|---|
| Cap9-lib2-2 | Cap9-lib2- | AACTTACAATCGGCTAATACTGCACCCCAGACACAAACTGTTCAAA |
| | F | ACCAAGGAATACTTC |
| | Cap9-R | GTTTATTGATTAACAAGCAATTACAGATTACGAGTCAGGT |
| Cap9-lib7-2 | Cap9-lib7- | AACCTGCAATCGGCAAACACCGCTCCTCAAACAGGGACCGTTCAA |
| | F | AACCAAGGAATACTT |
| | Cap9-R | GTTTATTGATTAACAAGCAATTACAGATTACGAGTCAGGT |
| Cap9-lib31-2 | Cap9-lib31- | AATTTGCAAAACTCAAATACTGGTCCAACTACAGAAAATGTTCAAA |
| | F | ACCAAGGAATACTTC |
| | Cap9-R | GTTTATTGATTAACAAGCAATTACAGATTACGAGTCAGGT |
| Cap9-lib33-2 | Cap9-lib33- | AATCTCCAGAGCAGCAACACAGCCCCTGCGACCGGAACTGTTCAA |
| | F | AACCAAGGAATACTT |
| | Cap9-R | GTTTATTGATTAACAAGCAATTACAGATTACGAGTCAGGT |
| Cap9-lib43-2 | Cap9-lib43- | AACCTACAGTCGGCAAACACCGCTCCTCAAACGGGGACCGTTCAA |
| | F | AACCAAGGAATACTT |
| | Cap9-R | GTTTATTGATTAACAAGCAATTACAGATTACGAGTCAGGT |
| Cap9-lib11-2 | Lib-LP-F | GCATCTTTGAACAATAAATGATTTAAATCAGGTATGGCTGCCGATGG |
| | | TTATCT |
| | Cap9-R | AGTTCCTGTAGTTGGACCAGTGTTTGAGTTTTGCAAATTTGTGGCC |
| | | ACTTGTCCATAGG |
| Cap9-lib46-2 | Lib-LP-F | GCATCTTTGAACAATAAATGATTTAAATCAGGTATGGCTGCCGATGG |
| | | TTATCT |
| | Cap9-R | ATCTGCGGTAGCTGCTTGTGTGTTGCCGCTCTGGAGGTTTGTGGCC |
| | | ACTTGTCCATAGG |

### Example 2: Methods

### Cell Culture

HEK293T cells were purchased from ATCC (ATCC, Manassas, Va.).

HEK293T cells were maintained in complete medium containing DMEM (Gibco, Grand Island, N.Y.), 10% FBS (Corning, Manassas, Va.), 1% Anti-Anti (Gibco, Grand Island, N.Y.). HEK293T cells were grown in adherent culture using 15 cm dish (Corning, Corning, Calif.) in a humidified atmosphere at 37° C. in 5% CO₂ and were sub-cultured after treatment with trypsin-EDTA (Gibco, Grand Island, N.Y.) for 2-5 min in the incubator, washed and re-suspended in the new complete medium.

### Construction of AAV Plasm Ids

Plasmid pAAV-RC8 contains the Rep encoding sequences from AAV2 and Cap encoding sequences from AAV8. We generated a fragment that contains 5′ MluI and AAV's native promoter, upstream of the Rep2 gene in the pAAV-RC8 plasmid, by using the forward primer:

5′-TAAGCCAACTAGTGGAACCGGTGCGGCCGCACGCGTGGAGTTTAAG
CCCGAGTGAGCACGCAGGGTCTCCATTTTGAAGCGGGAGGTTTGAACGC
GCAGCCGCCATGCCGGGGTT-3′,
and
reverse primer:
5′-GAAGATAACCATCGGCAGCCATTTAATTAAACCTGATTTAAATCAT
TTATTGTTCAAAG-3′.

To substitute VR VIII sequence of wild type AAV8, we introduced Ndel and Xbal restriction sites into 1756 bp and 1790 bp of the type 8 capsule (Cap8) gene, so the Cap8 region was generated by high-fidelity PCR amplification of two DNA fragments from plasmid pAAV-RC8.

One fragment was produced by using the forward primer:

5′-CTTTGAACAATAAATGATTTAAATCAGGTTTAATTAAATGGCTGCCG
ATGGTTATCTTC-3′,
and
reverse primer:
5′-TTCCAATTTGAGGAGCCGTGTTTTGCTGCTGCAACATATGGTTATCT
GCCACGATACCGTATT-3′;
the other fragment was produced by using the
forward primer:
5′-ACACGGCTCCTCAAATTGGAATCTAGACTGTCAACAGCCAGGGGGCC
TTACCCGGTATGGTCTG-3′,
and
reverse primer:
5′-GCCAACTCCATCACTAGGGGTTCCTGCGGCCGCTCGGTCCGCACGT
GGTTACCTACAAAATGCTAGCTTACAGATTACGGGTGAGGTAACG-3′.

Plasmid pssAAV-CMV-GFP-mut was digested by NotI (NEB, Ipswich, Mass.). The three fragments and linearized vector (pssAAV-CMV-GFP-mut) were assembled together with the NEB HiFi Builder (NEB, Ipswich, Mass.). The assembled product with the correct orientation and sequence was called pITR2-Rep2-Cap8-ITR2.

We then synthesized these 52 VR VIII oligo sequences with flanking 20nt overlapping sequences the same as Cap8 gene (Genewiz). These 52 sequences were used to substitute the VR VIII of AAV8 capsid backbone, individually, which were further subcloned into an all-in-one construct containing the modified capsid sequences with rep and inverted terminated repeats (ITRs) from AAV2 (FIG. 1A). Plasmid pITR2-Rep2-Cap8-ITR2 was digested with the enzyme Ndel (NEB, Ipswich, Mass.) and Xbal (NEB, Ipswich, Mass.) for linearization to generate a vector backbone. To substitute the wild type AAV8 VR VIII region, each VR VIII oligo was assembled with linearized pITR2-Rep2-Cap8-mut-ITR2 vector individually. The assembled product with the correct orientation and sequence was called pITR2-Rep2-Cap8-library-ITR2. Therefore, we have generated 52 different pITR2-Rep2-Cap8-library-ITR2 plasmids.

To generate recombinant pAAV-RC8-library plasmids, the whole Cap8-library fragment, 2.2 kb, from selected pITR2-Rep2-Cap8-library-ITR2 plasmids and backbone from pAAV-RC8, 5.2 kb, were assembled together using the NEB HiFi Builder (NEB, Ipswich, Mass.). Briefly, the whole Cap8-library region was produced by high-fidelity PCR amplification of plasmid pITR2-Rep2-Cap8-library-ITR2 using the forward primer 5′-GCATCTTTGAACAATAAATGATTTAAATCAGGTATGGCTGCCGATGGTTAT CT-3′ and reverse primer 5′-GTTTATTGATTAACAAGCAATTACAGATTACGGGTGAGGT-3′. The vector backbone was produced by high-fidelity PCR amplification of plasmid pAAV-RC8 using the forward primer 5′-TTGCTTGTTAATCAATAAACCG-3′ and reverse primer 5′-ACCTGATTTAAATCATTTATTGTTCAAAGATGC-3′. The assembled product with the correct orientation and sequence was called pAAV-RC8-library.

Plasmid pAAV-RC9 contains the Rep encoding sequences from AAV2 and Cap encoding sequences from AAV9, synthesized by Genewiz. The whole Cap9-library region was produced by high-fidelity PCR amplification of two DNA fragments from plasmid pAAV-RC9. One fragment was produced by using the primer sets in the Table 4, the other fragment was produced by using the primer sets in the Table 5. The linear vector backbone of pAAV-RC9 was also produced by high-fidelity PCR amplification of plasmid pAAV-RC9 using the forward primer of 5′-TTGCTTGTTAATCAATAAACCG-3′ and reverse primer of 5′-ACCTGATTTAAATCATTTATTGTTCAAAGATGC-3′. The two DNA fragments and linearized vector (pAAV-RC9) were assembled together using NEB HiFi Builder (NEB, Ipswich, Mass.). The product with the correct orientation and sequence was called pAAV-RC9-library.

TABLE 6
The AAV variants bearing 52 unique VR VIII DNA sequences. The mutations in reference
to the VR VIII of AA V8 were marked in red. The WT AAV8 VR VIII, which we named
AAV8-Lib40, were marked in blue.

| | | Protein_seq | |
|---|---|---|---|
| Variant | | (585-597/8, VPI | SEQ ID |
| name | Coding_DNA (1753-1791/4, VP1 numbering) | numbering) | No. |
| WT AAV8 | TAACTTGCAGCAGCAAAACACGGCTCCTCAAATTGGAACT | NLQQQNTAPQIGT | 2 |
| AAV8-Lib1 | TAACAATCAGAATACCAACACGGCTCCTACTGCAGGAACC | NNQNTNTAPTAGT | 3 |
| AAV8-Lib2 | TAACTTACAATCGGCTAATACTGCACCCCAGACACAAACT | NLQSANTAPQTQT | 4 |
| AAV8-Lib3 | TAACCTACAGCAGCAAAACACCGCTCCTACTGTGGGGGCC | NLQQQNTAPTVGA | 5 |
| AAV8-Lib4 | TAACAACCAGGCCGCTAACACGCAGGCGCAAACTGGACTT | NNQAANTQAQTGL | 6 |
| AAV8-Lib5 | TAACCTTCAAAGCGGCAACACACAAGCAGCTACCTCAGAT | NLQSGNTQAATSD | 7 |
| AAV8-Lib6 | TAATCAAAACAATGGAGCCAGTCAGACTCCTACGGCTTCCGAC | NQNNGASQTPTASD | 8 |
| AAV8-Lib7 | TAACCTGCAATCGGCAAACACCGCTCCTCAAACAGGGACC | NLQSANTAPQTGT | 9 |
| AAV8-Lib8 | TAATCTGCAGCAGACCAACTCGGCTCCCATTGTGGGGGCA | NLQQTNSAPIVGA | 10 |
| AAV8-Lib9 | TAACCTCCAGAGCGGCAACACACAAGCAGCTACTGCAGAT | NLQSGNTQAATAD | 11 |
| AAV8-Lib10 | TAATCTCCAGAGCAGCAGCACAGACCCTGCGACCGGAGAT | NLQSSSTDPATGD | 12 |
| AAV8-Lib11 | TAATTTGCAAAACTCAAACACTGGTCCAACTACAGGAACT | NLQNSNTGPTTGT | 13 |
| AAV8-Lib12 | TAACTTGCAGAGCTCAAATACAGCTCCCGCGACTGGAACT | NLQSSNTAPATGT | 14 |
| AAV8-Lib13 | TAACAACCAGGCCGCCAATACGCAGGCGCAGACCGGACTC | NNQAANTQAQTGL | 6 |
| AAV8-Lib14 | TAACCTCCAGAGCGGCAACACACAAGCATCTACTGCAGAT | NLQSGNTQASTAD | 15 |
| AAV8-Lib15 | TAACAACCAGTCCGCCAATACGCAGGCGCAGACCGGACTC | NNQSANTQAQTGL | 16 |
| AAV8-Lib16 | TAATTTGCAAAACTCAAATACTGCTGCAAGTACTGAAACT | NLQNSNTAASTET | 17 |
| AAV8-Lib17 | TAACCTGCAATCGGCCAACACCGCTCCTGCTACAGGGACC | NLQSANTAPATGT | 18 |
| AAV8-Lib18 | TAACCTACAGCAGCAAGACACCGCTCCTATTGTGGGGGCC | NLQQQDTAPIVGA | 19 |
| AAV8-Lib19 | TAATAATCAGAATGCTACAACTGCTCCCATAACCGGCAAC | NNQNATTAPITGN | 20 |
| AAV8-Lib20 | TAACCTGCAATCGTCTACGGCCGGACCCCAGACACAGACT | NLQSSTAGPQTQT | 21 |
| AAV8-Lib21 | TAACCTACAGCAGCAAAACACCGCTCCTATTGTGGGGGCC | NLQQQNTAPIVGA | 22 |
| AAV8-Lib22 | TAATTTGCAAGACTCAAATACTGGTCCAACTACTGGAACT | NLQDSNTGPTTGT | 23 |
| AAV8-Lib23 | TAATCTGCAGCAGACCAACTCAGCTCCCATTGTGGGGGCA | NLQQTNSAPIVGA | 10 |
| AAV8-Lib24 | TAACCTGCAGGCGGCTAACACTGCAGCCCAGACACAAGTT | NLQAANTAAQTQV | 24 |
| AAV8-Lib25 | TAACCTCCAGAGCGGCAACACACGAGCAGCTACCTCAGAT | NLQSGNTRAATSD | 25 |
| AAV8-Lib26 | TTACCTCCAGAGCGGCAACACACAAGCAGCTACCTCAGAT | YLQSGNTQAATSD | 26 |
| AAV8-Lib27 | TAATTTGCAAAACTCAAATACTGGTCCAACTACTGGAACT | NLQNSNTGPTTGT | 13 |
| AAV8-Lib28 | TAATTTGCAAAACTCAAACACTGGTCCAACTACTGGAACT | NLQNSNTGPTTGT | 13 |
| AAV8-Lib29 | TAACCTTCAGAGCAGCAACACACAAGCAGCTACCTCAGAT | NLQSSNTQAATSD | 27 |
| AAV8-Lib30 | TAACCTCCAGAGAGGCAACAGACAAGCAGCTACCGCAGAT | NLQRGNRQAATAD | 28 |
| AAV8-Lib31 | TAATTTGCAAAACTCAAATACTGGTCCAACTACAGAAAAT | NLQNSNTGPTTEN | 29 |
| AAV8-Lib32 | TAACAAACAGGACAGCAGTACACAGGCGACGACCGCGATT | NKQDSSTQATTAI | 30 |
| AAV8-Lib33 | TAATCTCCAGAGCAGCAACACAGCCCCTGCGACCGGAACT | NLQSSNTAPATGT | 14 |
| AAV8-Lib34 | TAATCTACAAAGCTCGGCAGAAACAGCCGAGACCGAAAGA | NLQSSAETAETER | 31 |
| AAV8-Lib35 | TAACCTACAGCAGCAAAACACCGCTCCTCAAATAGGGACC | NLQQQNTAPQIGT | 2 |
| AAV8-Lib36 | TAACTTGCAGCAAACCAATACAGGGCCTATTGTGGGAAAT | NLQQTNTGPIVGN | 32 |
| AAV8-Lib37 | TAACTTGCAGCAAACCAATACGGGGCCTATTGTGGGAAAT | NLQQTNTGPIVGN | 32 |
| AAV8-Lib38 | TAACCACCAGAGTGCCCAAGCACAGGCGCAGACCGGCTGG | NHQSAQAQAQTGW | 33 |
| AAV8-Lib39 | TAACCTGCAGGGCGGCAACACACAAGCAGCTACCGCAGAT | NLQGGNTQAATAD | 34 |
| AAV8-Lib40 | TAACTTGCAGCAGCAAAACACGGCTCCTCAAATTGGAACT | NLQQQNTAPQIGT | 2 |
| AAV8-Lib41 | TAACCTGCAGCAGACCAACGGAGCTCCCATTGTGGGAACT | NLQQTNGAPIVGT | 35 |
| AAV8-Lib42 | TAACCTGCAGTCGTCTACAGCCGGGCCTCAATCACAGACT | NLQSSTAGPQSQT | 36 |
| AAV8-Lib43 | TAACCTACAGTCGGCAAACACCGCTCCTCAAACGGGGACC | NLQSANTAPQTGT | 9 |
| AAV8-Lib44 | TAATTTGCAAAACTCAAATACTGCTCCGAGTACTGGAACT | NLQNSNTAPSTGT | 37 |
| AAV8-Lib45 | TAATTTCCAGAGCAGCAGCACAGACCCTGCGACCGGAGAT | NFQSSSTDPATGD | 38 |
| AAV8-Lib46 | TAACCTCCAGAGCGGCAACACACAAGCAGCTACCGCAGAT | NLQSGNTQAATAD | 11 |
| AAV8-Lib47 | TAACTTCCAAAACAATACAACAGCAGCAGATACAGAGATG | NFQNNTTAADTEM | 39 |
| AAV8-Lib48 | TAACCTCCAGAGCGGCAACACACAAGCAGCTACCTCAGAT | NLQSGNTQAATSD | 7 |
| AAV8-Lib49 | TAATTTACAAGCGGCCAATACTGCAGCCCAGACACAAGTT | NLQAANTAAQTQV | 24 |
| AAV8-Lib50 | TAACTTGCAGCAAGCCAATACAGGGCCTATTGTGGGAAAT | NLQQANTGPIVGN | 40 |
| AAV8-Lib51 | TAACCATCAGAGTCAGAACACCACAGCTTCCTATGGAAGT | NHQSQNTTASYGS | 41 |
| AAV8-Lib52 | TAACCTGCAACAGCAAAACGCCGCTCCTATTGTAGGGGCC | NLQQQNAAPIVGA | 42 |

### AAV Capsid Library Packaging

The packaging and purification of AAV capsid library were performed as previously described with some modifications. Briefly, HEK293T cells were co-transfected with 23.7 μg of individual pITR2-Rep2-Cap8-library-ITR2 plasmid and 38.7 μg of pHelper (Cell Biolabs) for separate packaging. Polyethyleneimine (PEI, linear, MW 25000, Polysciences, Inc., Warrington, Pa.) was used as transfection reagent. Cells were harvested 72 hrs post-transfection using cell lifter (Fisher Scientific, China), subjected to 3 rounds of freeze-thaw to recover the AAV variants inside the cells. The cell lysates were then digested with Benzonase (EMD Millipore, Denmark, Germany) and subjected to tittering by SYBR Green qPCR (Applied Biosystems, Woolston Warrington, UK) using primers specific to the Rep gene (forward: 5′-GCAAGACCGGATGTTCAAAT-3′, reverse: 5′-CCTCAACCACGTGAT CCTTT-3′). 5×10⁹ vg of each AAV variants were then mixed together. The mixture was then purified on iodixanol gradient (Sigma, St. Louis, Mo.) in Quick-Seal Polypropylene Tube (Beckman Coulter, Brea, Calif.) followed by ion exchange chromatography using HiTrap Q HP (GE Healthcare, Piscataway, N.J.). The elution was concentrated by centrifugation using centrifugal spin concentrators with 150K molecular-weight cutoff (MWCO) (Orbital biosciences, Topsfield, Mass.). Following purification, the mixture containing 52 AAV VR VIII variants was quantified again by qPCR using the primer sets for Rep gene and diluted into two parts. The first part contains three independent aliquots acting as control viral mixture before selection. The second part was used for tail vein injection into C57BL/6J mice, at 2.5×10¹¹ vg per animal, for in vivo selection.

When packaging rAAV-luciferase and rAAV-hFIX vectors, HEK293T cells were co-transfected with: i) pAAV-RC8 or selected pAAV-RC8-library and pAAV-RC9-library plasmids; ii) pAAV-CMV-Luciferase or pAAV-TTR-hFIX, respectively; iii) pHelper in equimolar amounts for each packaging. Plasmids were prepared using EndoFree Plasmid Kit (Qiagen, Hilder, Germany). The transfection, viral harvesting and purification steps were the same as the packaging of AAV VR VIII variants as mentioned above. The genome titer of the rAAV-luciferase vectors were quantified by qPCR using primers specific to the CMV promoter (forward: 5′-TCCCATAGTAACGCCAATAGG-3′, reverse: 5′-CTTGGCATATGATACACTTGATG-3′). The genome titer of the rAAV-hFIX vectors were quantified by qPCR using primers specific to the TTR promoter (forward: 5′-TCCCATAGTAACGCCAATAGG-3′, reverse: 5′-CTTGGCATATGATACACTTGATG-3′). The physical titer of rAAV8- and rAAV9-luciferase vectors were evaluated as described below (data not shown). The purity of rAAV were evaluated by SDS-PAGE silver staining, vector with ˜90% purity was used in our study (data not shown).

### In Vivo Selection for Liver-Targeting Variants

All animal work was performed in accordance with institutional guidelines under the protocols approved by the institutional animal care and use committee of WuXi AppTec (Shanghai). The C57BL/6J mice (Shanghai SLAC Laboratory Animal Co., Ltd.), male, 6 to 8-week-old, were tail vein injected with mixture of AAV VR VIII variants as described above. At week 1, 2 and 4 post-injection, the animals were euthanized by cervical dislocation after being anesthetized with isoflurane. For week 1 and 2, liver and brain were harvested, and for week 4, lung, liver, spleen, heart, kidney, lymph node, quadriceps muscle and brain were also harvested. Then the total DNA was extracted using DNeasy Blood & Tissue Kit (QIAGEN) according to the manufacturer's protocol and then analyzed by next generation sequencing to compare the AAV read counts after selection vs before selection.

TABLE 7
The list of AAV8 VR VIII variants selected for further in vitro and in vivo validation.
The variant name their VR VIII sequence in DNA and AA were showed. The mutations in
reference to the VR VIII of AAV8 were marked in red.

| | | | SEQ |
|---|---|---|---|
| | | Protein_seq (585-597, | ID |
| Variant name | Coding_dna (1753-1791, VP1 numbering) | VP1 numbering) | NO |
| WT AAV8 | AACTTGCAGCAGCAAAACACGGCTCCTCAAATTGGAACT | NLQQQNTAPQIGT | 2 |
| AAV8-Lib20 | AACCTGCAATCGTCTACGGCCGGACCCCAGACACAGACT | NLQSSTAGPQTQT | 21 |
| AAV8-Lib25 | AACCTCCAGAGCGGCAACACACGAGCAGCTACCTCAGAT | NLQSGNTRAATSD | 25 |
| AAV8-Lib43 | AACCTACAGTCGGCAAACACCGCTCCTCAAACGGGGACC | NLQSANTAPQTGT | 9 |
| AAV8-Lib44 | AATTTGCAAAACTCAAATACTGCTCCGAGTACTGGAACT | NLQNSNTAPSTGT | 37 |
| AAV8-Lib45 | AATTTCCAGAGCAGCAGCACAGACCCTGCGACCGGAGAT | NFQSSSTDPATGD | 38 |

TABLE 8
The AAV variants bearing 52 unique VR VIII DNA sequences. The mutations in
reference to the VR VIII of AAV9 were marked in red. The WT AAV9 VR VIII, which
we named AAV9-Lib38, were marked in blue.

| | | Protein_seq | SEQ |
|---|---|---|---|
| Variant | | (583-595/6, VP1 | ID |
| name | Coding_DNA (1752-1791/4, VP1 numbering) | numbering) | No. |
| WT AAV9 | AACCACCAGAGTGCCCAAGCACAGGCGCAGACCGGCTGG | NHQSAQAQAQTGW | 33 |
| AAV9-Lib1 | AACAATCAGAATACCAACACGGCTCCTACTGCAGGAACC | NNQNTNTAPTAGT | 3 |
| AAV9-Lib2 | AACTTACAATCGGCTAATACTGCACCCCAGACACAAACT | NLQSANTAPQTQT | 4 |
| AAV9-Lib3 | AACCTACAGCAGCAAAACACCGCTCCTACTGTGGGGGCC | NLQQQNTAPTVGA | 5 |
| AAV9-Lib4 | AACAACCAGGCCGCTAACACGCAGGCGCAAACTGGACTT | NNQAANTQAQTGL | 6 |
| AAV9-Lib5 | AACCTTCAAAGCGGCAACACACAAGCAGCTACCTCAGAT | NLQSGNTQAATSD | 7 |
| AAV9-Lib6 | AATCAAAACAATGGAGCCAGTCAGACTCCTACGGCTTCCGAC | NQNNGASQTPTASD | 8 |
| AAV9-Lib7 | AACCTGCAATCGGCAAACACCGCTCCTCAAACAGGGACC | NLQSANTAPQTGT | 9 |
| AAV9-Lib8 | AATCTGCAGCAGACCAACTCGGCTCCCATTGTGGGGGCA | NLQQTNSAPIVGA | 10 |
| AAV9-Lib9 | AACCTCCAGAGCGGCAACACACAAGCAGCTACTGCAGAT | NLQSGNTQAATAD | 11 |
| AAV9-Lib10 | AATCTCCAGAGCAGCAGCACAGACCCTGCGACCGGAGAT | NLQSSSTDPATGD | 12 |
| AAV9-Lib11 | AATTTGCAAAACTCAAACACTGGTCCAACTACAGGAACT | NLQNSNTGPTTGT | 13 |
| AAV9-Lib12 | AACTTGCAGAGCTCAAATACAGCTCCCGCGACTGGAACT | NLQSSNTAPATGT | 14 |
| AAV9-Lib13 | AACAACCAGGCCGCCAATACGCAGGCGCAGACCGGACTC | NNQAANTQAQTGL | 6 |
| AAV9-Lib14 | AACCTCCAGAGCGGCAACACACAAGCATCTACTGCAGAT | NLQSGNTQASTAD | 15 |
| AAV9-Lib15 | AACAACCAGTCCGCCAATACGCAGGCGCAGACCGGACTC | NNQSANTQAQTGL | 16 |
| AAV9-Lib16 | AATTTGCAAAACTCAAATACTGCTGCAAGTACTGAAACT | NLQNSNTAASTET | 17 |
| AAV9-Lib17 | AACCTGCAATCGGCCAACACCGCTCCTGCTACAGGGACC | NLQSANTAPATGT | 18 |
| AAV9-Lib18 | AACCTACAGCAGCAAGACACCGCTCCTATTGTGGGGGCC | NLQQQDTAPIVGA | 19 |
| AAV9-Lib19 | AATAATCAGAATGCTACAACTGCTCCCATAACCGGCAAC | NNQNATTAPITGN | 20 |
| AAV9-Lib20 | AACCTGCAATCGTCTACGGCCGGACCCCAGACACAGACT | NLQSSTAGPQTQT | 21 |
| AAV9-Lib21 | AACCTACAGCAGCAAAACACCGCTCCTATTGTGGGGGCC | NLQQQNTAPIVGA | 22 |
| AAV9-Lib22 | AATTTGCAAGACTCAAATACTGGTCCAACTACTGGAACT | NLQDSNTGPTTGT | 23 |
| AAV9-Lib23 | AATCTGCAGCAGACCAACTCAGCTCCCATTGTGGGGGCA | NLQQTNSAPIVGA | 10 |
| AAV9-Lib24 | AACCTGCAGGCGGCTAACACTGCAGCCCAGACACAAGTT | NLQAANTAAQTQV | 24 |
| AAV9-Lib25 | AACCTCCAGAGCGGCAACACACGAGCAGCTACCTCAGAT | NLQSGNTRAATSD | 25 |
| AAV9-Lib26 | TACCTCCAGAGCGGCAACACACAAGCAGCTACCTCAGAT | YLQSGNTQAATSD | 26 |
| AAV9-Lib27 | AATTTGCAAAACTCAAATACTGGTCCAACTACTGGAACT | NLQNSNTGPTTGT | 13 |
| AAV9-Lib28 | AATTTGCAAAACTCAAACACTGGTCCAACTACTGGAACT | NLQNSNTGPTTGT | 13 |
| AAV9-Lib29 | AACCTTCAGAGCAGCAACACACAAGCAGCTACCTCAGAT | NLQSSNTQAATSD | 27 |
| AAV9-Lib30 | AACCTCCAGAGAGGCAACAGACAAGCAGCTACCGCAGAT | NLQRGNRQAATAD | 28 |
| AAV9-Lib31 | AATTTGCAAAACTCAAATACTGGTCCAACTACAGAAAAT | NLQNSNTGPTTEN | 29 |
| AAV9-Lib32 | AACAAACAGGACAGCAGTACACAGGCGACGACCGCGATT | NKQDSSTQATTAI | 30 |
| AAV9-Lib33 | AATCTCCAGAGCAGCAACACAGCCCCTGCGACCGGAACT | NLQSSNTAPATGT | 14 |
| AAV9-Lib34 | AATCTACAAAGCTCGGCAGAAACAGCCGAGACCGAAAGA | NLQSSAETAETER | 31 |
| AAV9-Lib35 | AACCTACAGCAGCAAAACACCGCTCCTCAAATAGGGACC | NLQQQNTAPQIGT | 2 |
| AAV9-Lib36 | AACTTGCAGCAAACCAATACAGGGCCTATTGTGGGAAAT | NLQQTNTGPIVGN | 32 |
| AAV9-Lib37 | AACTTGCAGCAAACCAATACGGGGCCTATTGTGGGAAAT | NLQQTNTGPIVGN | 32 |
| AAV9-Lib38 | AACCACCAGAGTGCCCAAGCACAGGCGCAGACCGGCTGG | NHQSAQAQAQTGW | 33 |
| AAV9-Lib39 | AACCTGCAGGGCGGCAACACACAAGCAGCTACCGCAGAT | NLQGGNTQAATAD | 34 |
| AAV9-Lib40 | AACTTGCAGCAGCAAAACACGGCTCCTCAAATTGGAACT | NLQQQNTAPQIGT | 2 |
| AAV9-Lib41 | AACCTGCAGCAGACCAACGGAGCTCCCATTGTGGGAACT | NLQQTNGAPIVGT | 35 |
| AAV9-Lib42 | AACCTGCAGTCGTCTACAGCCGGGCCTCAATCACAGACT | NLQSSTAGPQSQT | 36 |
| AAV9-Lib43 | AACCTACAGTCGGCAAACACCGCTCCTCAAACGGGGACC | NLQSANTAPQTGT | 9 |
| AAV9-Lib44 | AATTTGCAAAACTCAAATACTGCTCCGAGTACTGGAACT | NLQNSNTAPSTGT | 37 |
| AAV9-Lib45 | AATTTCCAGAGCAGCAGCACAGACCCTGCGACCGGAGAT | NFQSSSTDPATGD | 38 |
| AAV9-Lib46 | AACCTCCAGAGCGGCAACACACAAGCAGCTACCGCAGAT | NLQSGNTQAATAD | 11 |
| AAV9-Lib47 | AACTTCCAAAACAATACAACAGCAGCAGATACAGAGATG | NFQNNTTAADTEM | 39 |
| AAV9-Lib48 | AACCTCCAGAGCGGCAACACACAAGCAGCTACCTCAGAT | NLQSGNTQAATSD | 7 |
| AAV9-Lib49 | AATTTACAAGCGGCCAATACTGCAGCCCAGACACAAGTT | NLQAANTAAQTQV | 24 |
| AAV9-Lib50 | AACTTGCAGCAAGCCAATACAGGGCCTATTGTGGGAAAT | NLQQANTGPIVGN | 40 |
| AAV9-Lib51 | AACCATCAGAGTCAGAACACCACAGCTTCCTATGGAAGT | NHQSQNTTASYGS | 41 |
| AAV9-Lib52 | AACCTGCAACAGCAAAACGCCGCTCCTATTGTAGGGGCC | NLQQQNAAPIVGA | 42 |

TABLE 9
The list of AAV9 variants selected for further in vitro and in vivo validation.
The variant name their VR VIII sequence in DNA and AA were showed. The
mutations in reference to the VR VIII of AAV9 were marked in red.

| | | Protein_seq | SEQ |
|---|---|---|---|
| Variant | | (583-595/6, | ID |
| name | Coding_dna (1747-1785, VP1 numbering) | VPI numbering) | NO |
| WT AAV9 | AACCACCAGAGTGCCCAAGCACAGGCGCAGACCGGCTGG | NHQSAQAQAQTGW | 33 |
| AAV9-Lib2 | AACTTACAATCGGCTAATACTGCACCCCAGACACAAACT | NLQSANTAPQTQT | 4 |
| AAV9-Lib11 | AATTTGCAAAACTCAAACACTGGTCCAACTACAGGAACT | NLQNSNTGPTTGT | 13 |
| AAV9-Lib31 | AATTTGCAAAACTCAAATACTGGTCCAACTACAGAAAAT | NLQNSNTGPTTEN | 29 |
| AAV9-Lib33 | AATCTCCAGAGCAGCAACACAGCCCCTGCGACCGGAACT | NLQSSNTAPATGT | 14 |
| AAV9-Lib43 | AACCTACAGTCGGCAAACACCGCTCCTCAAACGGGGACC | NLQSANTAPQTGT | 9 |
| AAV9-Lib46 | AACCTCCAGAGCGGCAACACACAAGCAGCTACCGCAGAT | NLQSGNTQAATAD | 11 |

### Next Generation Sequencing to Quantify the AAV Genome Reads in Tissues

The DNA from control viral mixture before injection and the total DNA isolated from various tissues were subjected to PCR to amplify the VR VIII region using the primer set (forward: 5′-CAAAATGCTGCCAGAGACAA-3′ and reverse: 5′-GTCCGTGTGAGGAATCTTGG-3′). The PCR products at the correct size were gel purified (Zymo Research, Irvine, Calif.) and then quantified by nanodrop. These products were analyzed by next generation sequencing with Illumina Hiseq X conducted at the WuXi NextCODE. During the analysis, the reads were separated by each VR VIII DNA sequence with no mismatch allowed. Then, we obtained the absolute read count of individual VR VIII in each experimental condition. Then, we converted the data into relative read count to normalize the difference for different time point and different tissues.

### Titration of AAV Particles by ELISA

The AAV particle concentration was determined by the Progen AAV8 Titration ELISA kit (Progen Biotechnik GMBH, Heidelberg, Germany), against a standard curve prepared in the ELISA kit. Briefly, the recombinant adeno-associated virus 8 reference standard stock (rAAV8-RSS, ATCC, VR-1816) and samples were diluted with ready-to-use sample buffer so that they can be measured within the linear range of the ELISA (7.81×10⁶-5.00×10⁸ capsids/mL). The rAAV8-RSS was diluted in the range of 1:2000 to 1:16000, whereas samples were diluted between 1:2000 and 1:256000. Pipette 100 μL of ready-to-use sample buffer (blank), serial dilutions of standard, and samples (both diluted in ready-to-use sample buffer) into the wells of the microtiter strips. Seal strips with adhesion foil provided and incubate for 1 h at 37° C. Next, the plate was emptied and washed with 200 μL ready-to-use sample buffer 3 times. Pipette 100 μL biotin conjugate into the wells and seal strips with adhesion foil. After a 1-hour incubation at 37° C., the plates were emptied and washed 3 times. 100 μL streptavidin conjugate was then added to the wells and incubated for 1 hour at 37° C. Repeat washing step as described above, and pipette 100 μL substrate into the wells. Incubate the plate for 15 minutes at room temperature, and stop color reaction by adding 100 μL of stop solution into each well. Measure intensity of color reaction with a photometer at 450 nm wavelength within 30 minutes.

### In Vitro Infectivity

HEK293T cells were seeded in 96-well cell-culture plates (Corning, Wujiang, JS) 16 hrs before transduction. Cells were mock infected or infected with rAAV-VR VIII variants individually, MOI=10,000, in serum- and antibiotic-free DMEM for 2 hrs. 48 hrs post infection, the cells were lysed to detect luciferase expression using the Bright-Glo™ Luciferase Assay System (Promega, Madison, Wis.) according to the manufacturer's instructions.

### Sodium Dodecyl Sulfate-Polyacrylamide

For sodium dodecyl sulfate-polyacrylamide gel electrophoresis (SDS-PAGE) analysis, samples were denatured in NuPage Reducing Agent and NuPAGE LDS Sample Buffer (both from Invitrogen, Cartsbad, Calif.) at 100° C. for 10 min before being loaded onto NuPAGE 4-12% Bis-Tris minigels (Invitrogen, Cartsbad, Calif.). After electrophoresis, gels were silver-stained, using a Fast Silver Stain Kit (Beyotime, Shanghai, China). View gels using a white light box and a suitable imaging system.

In Vivo rAAV-Luciferase and Serum ALT Detection

The C57BL/6J mice, male, 6 to 8-week-old, were injected with appropriate amount of rAAV-luciferase vectors by tail vein injection. Bioluminescence were detected at day 3, week 1 and week 2 after viral injection. Before each detection, the mice will receive the 15 mg/ml D-Luciferin (PerkinElmer) by intraperitoneal injection. 10 mins after D-Luciferin injection, the mice will receive anesthesia using isoflurane. Xenogen Lumina II small animal in vivo imaging system (PerkinElmer) was used to select the region of interest (ROI), quantify and analyze the signal presented as photons/second/cm2/steridian (p/sec/cm2/sr). After the bioluminescence detection at week 2, the animals will be euthanized using 10% CO₂ followed by serum and tissue collection for serum alanine aminotransferase (ALT) detection and genome copy number detection. The ALT levels was determined by Alanine Aminotransferase Activity Assay Kit (SIGMA) according to the manufacturer's protocol.

In Vivo rAAV-hFIX Transduction

The potency of rAAV-hFIX gene transfer efficiency was initially assessed in 6 to 8-week-old male wild-type C57BL/6J mice by assessing hFIX levels in plasma following tail vein injection of the vector. Next, the F9 KO mice in C57BL/6J background purchased from Shanghai Model Organisms, male, 6 to 8-week-old, were injected with appropriate amount of AAV vectors by tail vein injection to assess the efficacy.

### Tissue, Plasma and Serum Collection

At appropriate time after viral injection, blood was collected by retroorbital bleeding. For terminal blood withdraw, immediately after CO₂ euthanasia, cardiac puncture was performed to collect blood followed by perfusion using PBS to harvest livers. The largest liver lobe was fixed with 10% Neutral buffered formalin (NBF) for pathological examination. Two independent sampling of other liver lobes were collected for snap-frozen and maintained in −80° C. for genome copy number detection. For serum collection, blood is placed in 4° C. for 2 hrs. Then spin down the blood at 8000 rpm for 15 mins, and aspirate the supernatant. For plasma collection, blood was added into 3.8% sodium citrate at a ratio of 9:1. Then, spin down the mixture at 8000 rpm for 5 mins, and aspirate the supernatant. The serum and plasma were maintained in −80° C.

Detection of hFIX Expression and Activity

The hFIX expression level was determined by an enzyme-linked immunosorbent assay (ELISA) (Affinity Biologicals, Ancaster, ON, Canada) according to the manufacturer's protocol. Briefly, a flat-bottomed, 96-well plate was coated with goat antibody against human factor IX. Standards were made by using serial dilutions of calibrator plasma (0.0313-1 IU/mL). Mouse plasma was diluted 1:200 in sample diluent buffer, and 100 μL samples and standards were added to the wells. After a 1-hour incubation at room temperature, the plates were emptied and washed with 300 μL diluted wash buffer 3 times. The plates were then incubated for 30 minutes at r temperature with 100 μL horseradish peroxidase (HRP)—conjugated secondary antibody solution. After a final wash step, the HRP activity was measured with Tetramethylbenzidine (TMB) substrate. The color reaction was stopped after 10 minutes using stop solution and read spectrophotometrically at 450 nm within 30 minutes. The reference curve is a log-log plot of the absorbance values versus the factor IX concentration, and the factor IX content in plasma samples can be read from the reference curve.

The hFIX activity in mice was determined in a chromogenic assay using the ROX factor IX activity assay kit (Rossix, Molndal, Sweden) according to the manufacture's protocol. Briefly, standard dilutions were prepared using normal human plasma in diluent buffer, range from 25% to 200% activity (100% activity is defined as 1 IU/mL factor IX in plasma). The experimental plasma samples were diluted 1:320 in diluent buffer, and 25 μL samples and standards were added to low binding 96 well microplates. 25 μL Reagent A (containing lyophilized human factor VIII, human factor X, bovine factor V and a fibrin polymerization inhibitor) was added to the wells and incubated for 4 minutes at 37° C. And then 150 μL Reagent B (containing lyophilized human factor XIa, human factor II, calcium chloride and phospholipids) was added to the wells. After 8 minutes at 37° C., activated factor X generation was terminated by the addition of 50 μL factor Xa Substrate (Z-D-Arg-Gly-Arg-pNA), and the absorbance was read at 405 nm. Plot the maximal absorbance change/minute (ΔA405max/min) vs. factor IX activity in a Log-Log graph, and the factor IX activity of the samples can be calculated using the reference curve.

### In Vivo Viral Genome Copy Number

Absolute qPCR using SYBR Green (Applied Biosystems, Woolston Warrington, UK) was used to quantify AAV viral genome copy number. Total DNA was extracted from various tissues using DNeasy Blood & Tissue Kit (QIAGEN, Hilden, Germany) according to the manufacturer's protocol. Total DNA concentration was determined using Nanodrop, and 40 ng of DNA from each sample was used as the template for qPCR. qPCR was performed on all tissue samples and control, done in triplicate, using primers specific for the CMV promoter (forward: TCCCATAGTAACGCCAATAGG, reverse: CTTGGCATATGATACACTTGATG). Linearized pssAAV-CMV-luci-mut plasmid at 2.89×10¹, 2.89×10², 2.89×10³, 2.89×10⁴, 2.89×10⁵, 2.89×10⁶, 2.89×10⁷ copy numbers (0.0002, 0.002, 0.02, 0.2, 2, 20, 200 pg) were used to generate a standard curve to calculate the copy numbers.

### Example 3: In Vivo Selection

52 different VR VIII sequences (Table 6) their corresponding GenBank ID were summarized in Table 7. These 52 sequences were used to substitute the VR VIII of AAV8 capsid backbone, individually, which were further subcloned into an all-in-one construct containing the modified capsid sequences with rep and inverted terminated repeats (ITRs) from AAV2. These constructs were used to package wild-type-like AAV particles, individually, then mixed together at equal viral genome, followed by purification. The purified AAV variant library was divided into two parts. One part was used for NGS detection (n=3) as the starting library baseline. Another part was subjected to systemic delivery for in vivo selection to isolate liver and brain-targeting AAV variants (FIG. 1). Notably, the design contained all the available unique VR VIII sequences including that of WT AAV8, or AAV8-Lib40, in our screen (Table 6). We used AAV8-Lib40 as our internal control during the screening and selection process.

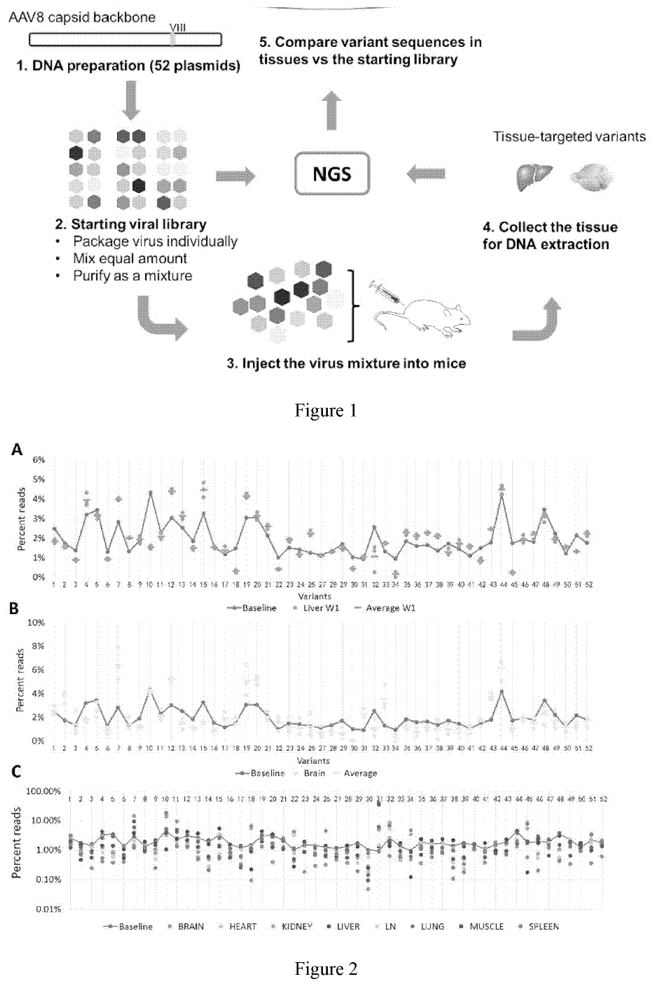

At week 1 post administration, liver and brain were harvested. Compared with the starting library and AAV8-Lib40, we were able to identify a few variants enriched in liver (FIG. 2A) and brain (FIG. 2B). At week 4 post administration, lung, liver, spleen, heart, kidney, lymph node, quadriceps (QA) muscle and brain were also harvested to evaluate biodistribution. We were able to identify variants that preferably target liver or brain than other tissues (FIG. 2C, Table 10).

TABLE 10
Variants that showed increased liver targeting, normalized to WT AAV8 to
baseline as 100%.

| | Protein_seq (585-597/8, | | Ratio (relative |
|---|---|---|---|
| Variants | VP1 numbering) | SEQ ID No. | to wtAAV8) |
| AAV8-Lib01 | NNQNTNTAPTAGT | 3 | 106.14% |
| AAV8-Lib04 | NNQAANTQAQTGL | 6 | 227.97% |
| AAV8-Lib05 | NLQSGNTQAATSD | 7 | 181.93% |
| AAV8-Lib07 | NLQSANTAPQTGT | 9 | 230.06% |
| AAV8-Lib08 | NLQQTNSAPIVGA | 10 | 116.07% |
| AAV8-Lib09 | NLQSGNTQAATAD | 11 | 112.02% |
| AAV8-Lib11 | NLQNSNTGPTTGT | 13 | 119.50% |
| AAV8-Lib12 | NLQSSNTAPATGT | 14 | 253.36% |
| AAV8-Lib13 | NNQAANTQAQTGL | 6 | 180.94% |
| AAV8-Lib15 | NNQSANTQAQTGL | 16 | 257.83% |
| AAV8-Lib19 | NNQNATTAPITGN | 20 | 239.08% |
| AAV8-Lib20 | NLQSSTAGPQTQT | 21 | 181.06% |
| AAV8-Lib21 | NLQQQNTAPIVGA | 22 | 150.83% |
| AAV8-Lib23 | NLQQTNSAPIVGA | 10 | 110.46% |
| AAV8-Lib25 | NLQSGNTRAATSD | 25 | 129.13% |
| AAV8-Lib33 | NLQSSNTAPATGT | 14 | 100.38% |
| AAV8-Lib35 | NLQQQNTAPQIGT | 2 | 131.97% |
| AAV8-Lib36 | NLQQTNTGPIVGN | 32 | 121.99% |
| AAV8-Lib37 | NLQQTNTGPIVGN | 32 | 131.34% |
| AAV8-Lib38 | NHQSAQAQAQTGW | 33 | 121.67% |
| AAV8-Lib40 | NLQQQNTAPQIGT | 2 | 100.00% |
| AAV8-Lib43 | NLQSANTAPQTGT | 9 | 142.69% |
| AAV8-Lib44 | NLQNSNTAPSTGT | 37 | 258.75% |
| AAV8-Lib46 | NLQSGNTQAATAD | 11 | 111.35% |
| AAV8-Lib47 | NFQNNTTAADTEM | 39 | 124.68% |
| AAV8-Lib48 | NLQSGNTQAATSD | 7 | 176.81% |
| AAV8-Lib49 | NLQAANTAAQTQV | 24 | 112.13% |
| AAV8-Lib52 | NLQQQNAAPIVGA | 42 | 128.43% |

While before the purification, we were able to titer all the AAV VR VIII variants individually for mixing equal amount and purification, we failed to detect AAV8-Lib26 by NGS both in our starting library and screens (FIG. 2A-C). This implied that the mutations in AAV8-Lib26 may not comply with the current AAV purification methods. Apart from this, we concluded that our capsid library design and screen strategy yielded highly viable AAV virions that facilitated the enrichment of liver- and brain-targeted variants.

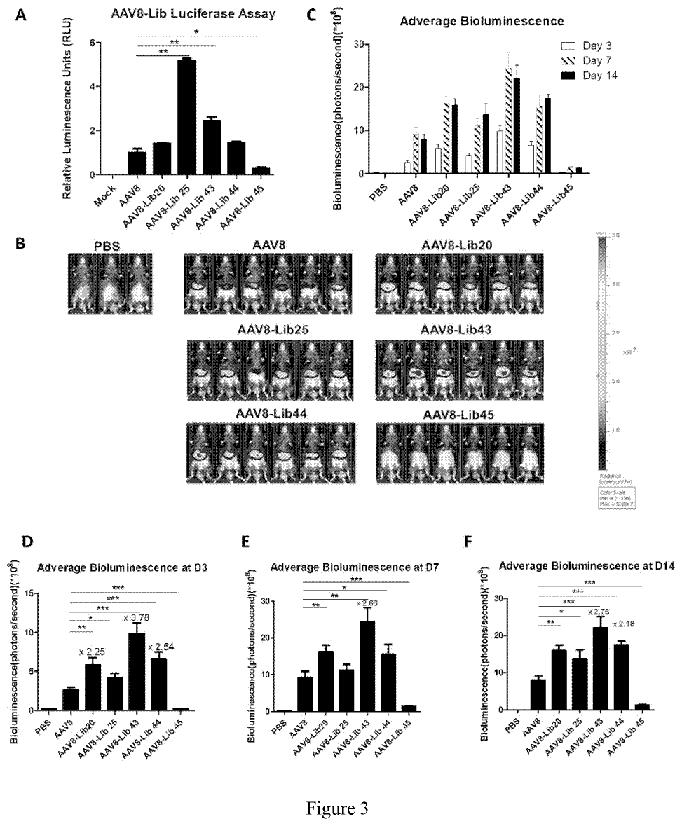

To further validate the gene delivery capability, the selected VR VIII sequences (Table 8) were subcloned into recombinant AAV capsid plasmid for the packaging of luciferase reporter gene. AAV8-Lib25 and AAV8-Lib43 demonstrated significantly higher transgene expression in vitro (FIG. 3A). Importantly, we found most of novel AAV variants showed highly significant increase in in vivo transduction (FIG. 3B-3E). As negative control, AAV8-Lib45, whose VR VIII was downregulated during our screen, showed significantly decreased transduction both in vitro (FIG. 3A) and in vivo (FIGS. 3B and 3C). These results, to an extent, validated our screen process and results.

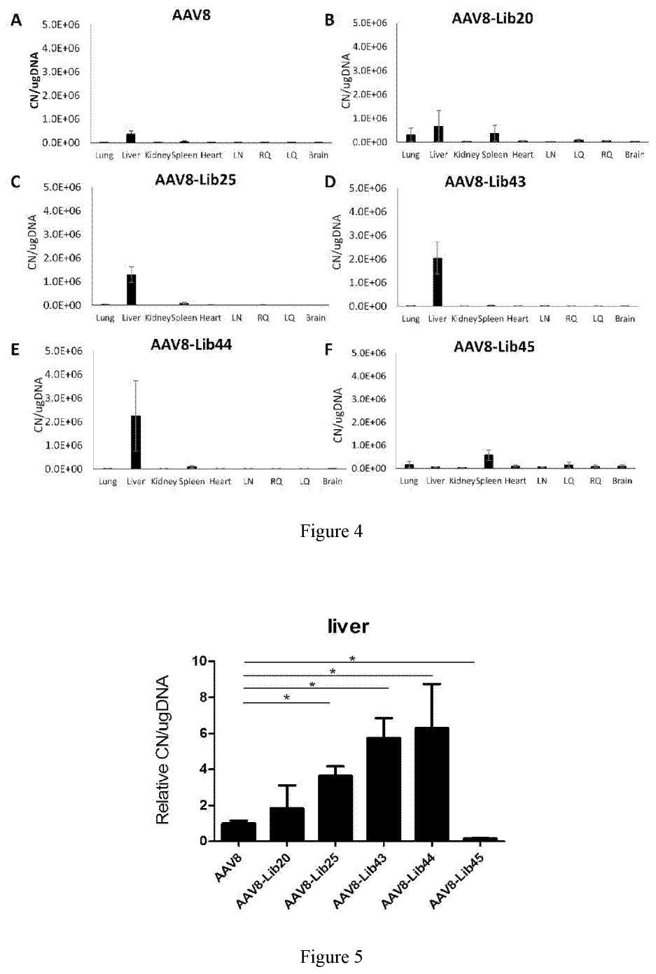

Furthermore, when we systemically characterized their biodistribution, we confirmed that these variants maintained a liver targeting profile as indicated by dominant GCNs in the liver than other tissues (FIG. 4A-E). Importantly, the liver genome copy numbers were significantly higher for AAV8 VR VIII variants than AAV8 (FIG. 5) further confirming the improved targeting capability. AAV8-Lib45, on the other hand, showed significantly lower liver GCNs further confirming our screen strategy (FIG. 5).

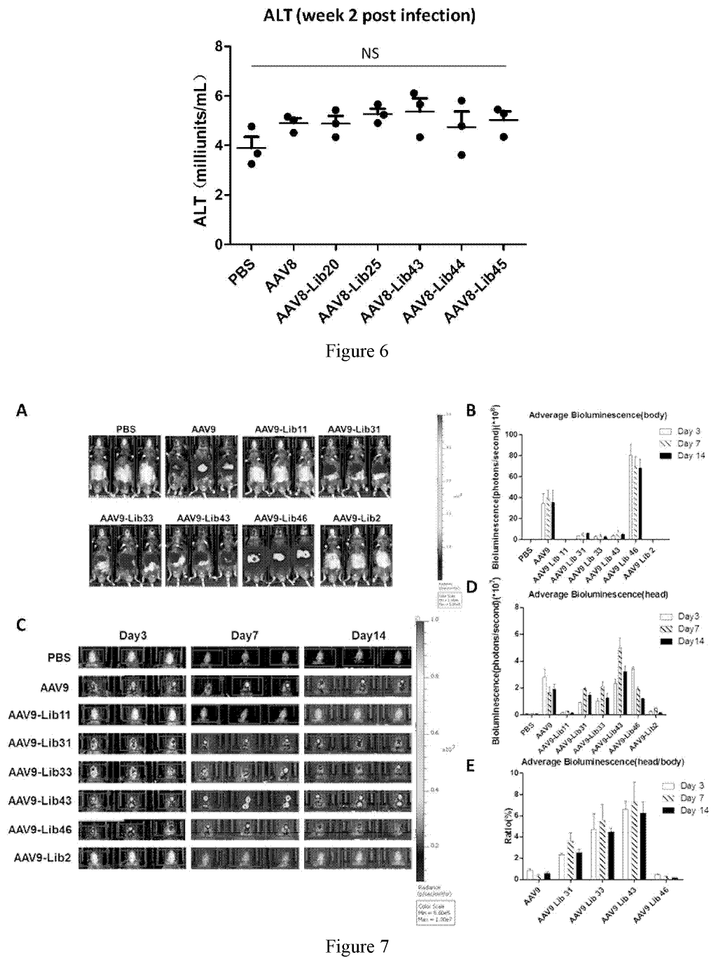

As a gene therapy vector, it is of most importance to have a good safety profile. To this end, we detected serum alanine transaminase (ALT) level, an important maker for liver toxicity. The ALT level was maintained below baseline for all of the groups (FIG. 6). These results indicate that AAV8 VRIIII variants could serve as alternative gene delivery tool to the liver.

As we have identified promising VR VIII sequences for gene delivery to the brain, we hypothesized that substituting WT VR VIII of AAV9 with brain-enriched VR VIII sequences (FIG. 2B) would generate variants with higher CNS-targeting capability. To test it, the AAV9-VR VIII capsid (Table 9) were used to package the genetic payload carrying luciferase reporter gene for evaluating transduction efficiency. We found that AAV9-Lib46 showed significantly higher transgene expression than WT AAV9 in vivo (FIGS. 7A and 7B). Interestingly, AAV9-Lib31, AAV9-Lib33, and in particular, AAV9-Lib43 showed a peripheral tissue-detargeting while maintained comparable CNS gene delivery (FIG. 7A). To this end, we specifically compare and qualify the luciferase expression in the head (FIGS. 7C and 7D) and found a dramatic shift for the head/body ratio of transgene expression (FIG. 7G).

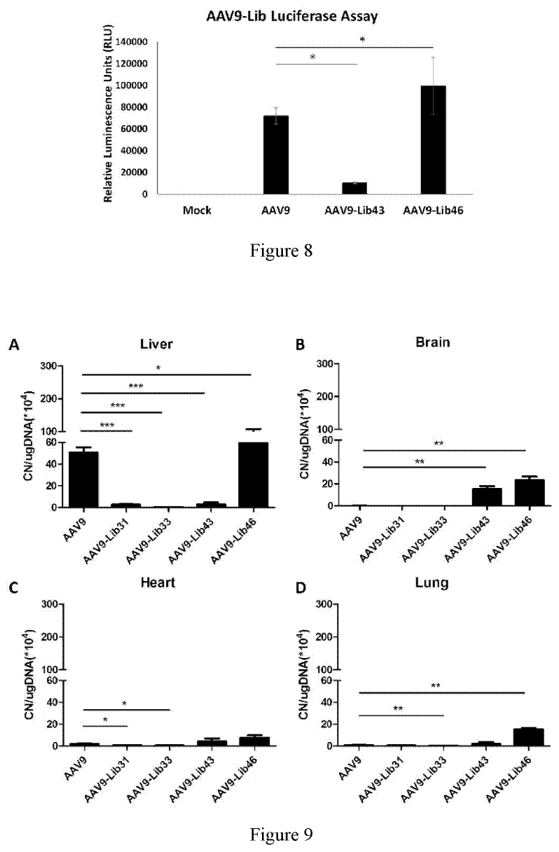

Then, we tested the in vitro transduction of our leading candidates AAV9-Lib43 and AAV9-Lib46. Following infection HEK293T cells, AAV9-Lib43 showed significantly decreased transgene expression (FIG. 8) and AAV9-Lib46 showed significantly increased transgene expression (FIG. 8). These data were consistent with their overall body expression in vivo (FIGS. 7A and 7B).

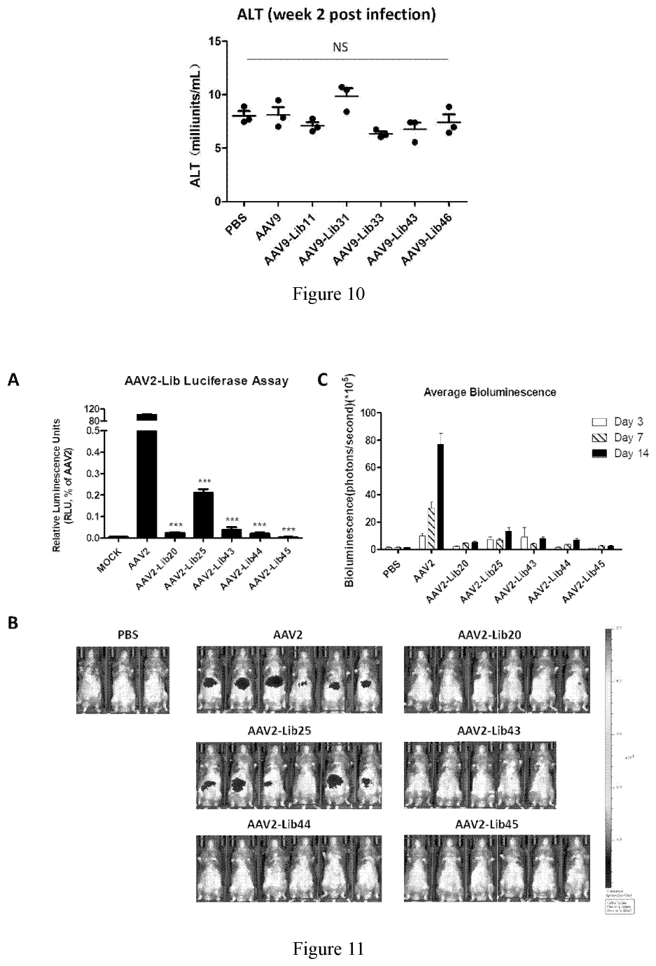

Next, we profiled the biodistribution of AAV9 and AAV9 VR VIII variants. As is well known that AAV9 has a tropism for liver, heart and CNS, we observed significantly decreased GCNs in the liver for AAV9-Lib31, AAV9-Lib33, and AAV9-Lib43 and higher GCNs for AAV9-Lib46 (FIG. 9A), consistent with the transgene expression results (FIG. 8A). AAV9-Lib43 and AAV9-Lib46 demonstrated significantly increased GCNs in the brain (FIG. 9B). Though not significant, we also observed elevated GCNs in heart and lung (FIGS. 9C and 9D). Furthermore, no ALT elevation were detected following AAV9 VR VIII variants—mediated gene delivery (FIG. 10). These results indicate that AAV9 VRIIII variants could serve as alternative gene delivery tool to the CNS following systemic gene delivery.

### Example 4: AAV2 VR VIII Variants

5 sequences listed in Table 11 were used to substitute the VR VIII of AAV2 capsid backbone (corresponding to amino acid position 582-594 of WT AAV2 YP_680426.1 (GenBank: NC_001401.2), individually, which were further subcloned into an all-in-one construct containing the modified capsid sequences with rep and inverted terminated repeats (ITRs) from AAV2. These constructs were used to package wild-type-like AAV particles, individually, then mixed together at equal viral genome, followed by purification. The purified AAV variant library was divided into two parts. One part was used for NGS detection (n=3) as the starting library baseline. Another part was subjected to systemic delivery for in vivo selection to isolate liver and brain-targeting AAV variants.

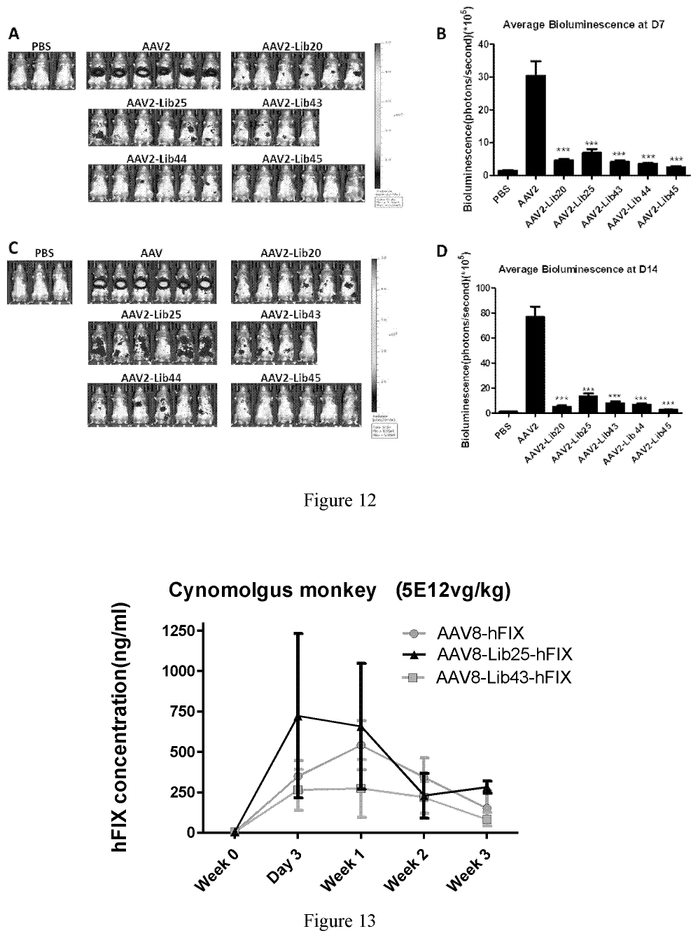

To further validate the gene delivery capability, the selected VR VIII sequences (Table 11) were subcloned into recombinant AAV2 capsid plasmid for the packaging of luciferase reporter gene. AAV2-Lib20, AAV2-Lib25, AAV2-Lib43, AAV2-Lib44, AAV2-Lib45 demonstrated significantly lower transgene expression in vitro (FIG. 11A). Importantly, we found most of novel AAV variants showed highly significant decrease in in vivo transduction (FIG. 11B-11C and FIG. 12A-D).

TABLE 11
The list of AAV2 VR VIII variants selected for further in vitro and in vivo
validation. The variant name their VR VIII sequence in DNA and AA were showed.
The mutations in reference to the VR VIII of AAV2 were marked in bold.

| Variant | | Protein_seq (585-597, |
|---|---|---|
| name | Coding_dna (1753-1791, VP1 numbering) | VP1 numbering) |
| WT AAV8 | AACCTCCAGAGAGGCAACAGACAAGCAGCTACCGCAGAT | NLQRGNRQAATAD |
| AAV2-Lib2 | AACCTGCAATCGTCTACGGCCGGACCCCAGACACAGACT | NLQSSTAGPQTQT |
| AAV2-Lib2 | AACCTCCAGAGCGGCAACACACGAGCAGCTACCTCAGAT | NLQSGNTRAATSD |
| AAV2-Lib4 | AACCTACAGTCGGCAAACACCGCTCCTCAAACGGGGACC | NLQSANTAPQTGT |
| AAV2-Lib4 | AATTTGCAAAACTCAAATACTGCTCCGAGTACTGGAACT | NLQNSNTAPSTGT |
| AAV2-Lib4 | AATTTCCAGAGCAGCAGCACAGACCCTGCGACCGGAGAT | NFQSSSTDPATGD |

### Example 5: Delivering a Nucleic Acid Vector to a Cell and/Tissue Using rAAV Used to Package a Genetic Payload that Comprise a Heterologous Nucleic Acid Region Comprising a Sequence Encoding a Protein or Polypeptide of Interest

The protein or polypeptide of interest is a protein or polypeptide describe in Table 12-14.

AAV8-hFIX, AAV8-Lib25-hFIX and AAV8-Lib43-hFIX were injected into 3-4 yeas old male cynomolgus monkeys with the dose of 5E12 vg/kg, monkeys enrolled in these experiments were all tested with neutralization antibody titer<1:50 against AAV8. Blood samples were harvested before dosage and at Day3, week1, week2 and week3, hFIX expression were detected in plasma by ELISA. The result shows that all of AAV8, AAV-Lib25 and AAV8-Lib43 can express hFIX efficiently in monkeys, AAV8-Lib25 express higher hFIX than AAV8 and AAV8-Lib43 (FIG. 13).

TABLE 12
Exemplary Proteins and polypeptides of interest (Liver Disease)

| | Non-limiting Exemplary diseases, | |
|---|---|---|
| Protein or Polypeptide | disorders, or phenotypes | Non-limiting NCBI Protein IDs |
| Cystathionine-beta-synthase | Homocystinuria | NP_000062.1, NP_001171479.1, |
| (CBS) | | NP_001171480.1 |
| Factor IX (FIX) | Hemophilia B | NP_000124.1 |
| Factor VIII (F8) | Haemophilia A | NP_000123.1, NP_063916.1 |
| Glucose-6-phosphatase catalytic | Glycogen Storage Disease Type I | NP_001257326.1 |
| subunit (G6PC) | (GSD1) | AAI30479.1 |
| | | AAI36370.1 |
| Glucose 6-phosphatase (G6Pase) | GSD-Ia | NP_000142.2, NP_001257326.1 |
| Glucuronidase, beta (GUSB) | MPSVII-Sly | NP_000172.2, NP_001271219.1 |
| Hemochromatosis (HFE) | Hemochromatosis | NP_000401.1, NP_620572.1, |
| | | NP_620573.1, NP_620575.1, |
| | | NP_620576.1, NP_620577.1, |
| | | NP_620578.1, NP_620579.1, |
| | | NP_620580.1 |
| Iduronate 2-sulfatase (IDS) | MPSII-Hunter | NP_000193.1, NP_001160022.1, |
| | | NP_006114.1 |
| Iduronidase, alpha-l (IDUA) | MPSI-Hurler | NP_000194.2, AAA51698.1 |
| Low density lipoprotein receptor | Phenylketonuria (PKU) | NP_000518.1, NP_001182727.1, |
| (LDLR) | | NP_001182728.1, NP_001182729.1, |
| | | NP_001182732.1, AAP36025.1 |
| Myophosphorylase (PYGM) | McArdle disease (glycogen storage | NP_001158188.1, NP_005600.1 |
| | disease type V, GSD5) | |
| N-acetylglucosaminidase, alpha | Sanfilippo syndrome (MPSIIIB) | NP_000254.2 |
| (NAGLU) | | |
| N-sulfoglucosamine | Mucopolysaccharidosis type IIIA | NP_000190.1 |
| sulfohydrolase (SGSH) | (MPS IIIA) | NP_001339851.1 |
| | | NP_001339850.1 |
| | | AAH47318.1 |
| Ornithine carbamoyltransferase | OTC deficiency | NP_000522.3, AAA59975.1 |
| (OTC) | | |
| Phenylalanine hydroxylase | Hypercholesterolaemia or | NP_000268.1 |
| (PAH) | Phenylketonuria (PKU) | |
| UDP glucuronosyltransferase 1 | Crigler-Najjar syndrome | NP_000454.1 |
| family, polypeptide A1 | | |
| (UGT1A1) | | |

TABLE 13
Exemplary Proteins and polypeptides of interest (CNS Disease)

| | Non-limiting Exemplary diseases, | Non-limiting NCBI Protein IDs or |
|---|---|---|
| Protein or Polypeptide | disorders, or phenotypes | Patent SEQ ID NOs |
| Acid alpha-glucosidase (GAA) | Pompe disease | NP_000143.2, NP_001073271.1, |
| | | NP_001073272.1 |
| ApaLI | Mitochondrial heteroplasmy, | YP_007161330.1 |
| | myoclonic epilepsy with ragged red | |
| | fibers (MERRF) or mitochondrial | |
| | encephalomyopathy, lactic acidosis, | |
| | and stroke-like episodes (MELAS) | |
| Aromatic L-amino acid | Parkinson's disease | NP_000781.1, NP_001076440.1, |
| decarboxylase (AADC) | | NP_001229815.1, NP_001229816.1, |
| | | NP_001229817.1, NP_001229818.1, |
| | | NP_001229819.1 |
| Aspartoacylase (ASPA) | Canavan's disease | NP_000040.1, NP_001121557.1 |
| Battenin | Ceroid lipofuscinosis neuronal 3 | NP_000077.1 |
| | (CLN3) | NP_001035897.1 |
| | | NP_001273033.1 |
| | | NP_001273034.1 |
| | | NP_001273038.1 |
| | | NP_001273039.1 |
| | | AAH04433.1 |
| Ceroid lipofuscinosis neuronal 2 | Late infantile neuronal | NP_000382.3, AAB80725.1 |
| (CLN2) | ceroidlipofuscinosis or Batten's | |
| | disease | |
| Cluster of Differentiation 86 | Malignant melanoma | NP_001193853.1, NP_001193854.1, |
| (CD86 or B7-2) | | NP_008820.3, NP_787058.4, |
| | | NP_795711.1 |
| Cystathionine-beta-synthase | Homocystinuria | NP_000062.1, NP_001171479.1, |
| (CBS) | | NP_001171480.1 |
| Dystrophin or Minidystrophin | Muscular dystrophy | NP_000100.3, NP_003997.1, |
| | | NP_004000.1, NP_004001.1, |
| | | NP_004002.3, NP_004003.2, |
| | | NP_004004.1, NP_004005.1, |
| | | NP_004006.1, NP_004007.1, |
| | | NP_004008.1, NP_004009.1, |
| | | NP_004010.1, NP_004011.2, |
| | | NP_004012.2, NP_004013.1, |
| | | NP_004014.2 |
| Frataxin (FXN) | Friedreich ataxia (FA) | NP_000135.2 |
| | | NP_852090.1 |
| | | AAH23633.1 |
| | | AAH48097.1 |
| Glial cell-derived neurotrophic | Parkinson's disease | NP_000505.1, NP_001177397.1, |
| factor (GDNF) | | NP_001177398.1, NP_001265027.1, |
| | | NP_954701.1 |
| Glutamate decarboxylase | Parkinson's disease | NP_000808.2, NP_038473.2 |
| 1(GAD1) | | |
| Glutamate decarboxylase 2 | Parkinson's disease | NP_000809.1, NP_001127838.1 |
| (GAD2) | | |
| Hexosaminidase A, α | Tay-Sachs | NP_000511.2 |
| polypeptide, also called beta- | | |
| Hexosaminidase alpha (HEXA) | | |
| Hexosaminidase B, β polypeptide, | Tay-Sachs | NP_000512.1, NP_001278933.1 |
| also called beta- Hexosaminidase | | |
| beta (HEXB) | | |
| Interleukin 12 (IL-12) | Malignant melanoma | NP_000873.2, NP_002178.2 |
| Methyl CpG binding protein 2 | Rett syndrome | NP_001104262.1, NP_004983.1 |
| (MECP2) | | |
| Myotubularin 1 (MTM1) | X-linked myotubular myopathy | NP_000243.1 |
| NADH ubiquinone | Leber hereditary optic | YP_003024035.1 |
| oxidoreductase subunit 4 (ND4) | | |
| Nerve growth factor (NGF) | Alzheimer's disease | NP_002497.2 |
| neuropeptide Y (NPY) | Parkinson's disease, epilepsy | NP_000896.1 |
| Neurturin (NRTN) | Parkinson's disease | NP_004549.1 |
| Palmitoyl-protein thioesterase 1 | Ceroid lipofuscinosis neuronal 1 | NP_000301.1 |
| (PPT1) | (CLN1) | AAH08426.1 |
| Sarcoglycan alpha, beta, gamma, | Muscular dystrophy | SGCA |
| delta, epsilon, or zeta (SGCA, | | NP_000014.1, NP_001129169.1 |
| SGCB, SGCG, SGCD, SGCE, or | | SGCB |
| SGCZ) | | NP_000223.1 |
| | | SGCG |
| | | NP_000222.1 |
| | | SGCD |
| | | NP_000328.2, |
| | | NP_001121681.1, |
| | | NP_758447.1 |
| | | SGCE |
| | | NP_001092870.1, |
| | | NP_001092871.1, |
| | | NP_003910.1 |
| | | SGCZ |
| | | NP_631906.2 |
| Tumor necrosis factor receptor | Arthritis, Rheumatoid arthritis | SEQ ID NO. 1 ofWO2013025079 |
| fused to an antibody Fc | | |
| (TNFR:Fc) | | |
| Ubiquitin-protein ligase E3A | Angelman Syndrome (AS) | NP_570853.1 |
| (UBE3A) | | NP_000453.2 |
| | | NP_570854.1 |
| | | NP_001341434.1 |
| | | AAH02582.2 |
| β-galactosidase 1 (GLB1) | GM1 gangliosidosis | NP_000395.3 |
| | | AAB81350.1 |

TABLE 14
Exemplary Proteins and polypeptides of interest (Other Disease)

| | Non-limiting Exemplary diseases, | Non-limiting NCBI Protein IDs or |
|---|---|---|
| Protein or Polypeptide | disorders, or phenotypes | Patent SEQ ID NOs |
| Adenine nucleotide translocator | progressive external | NP_001142.2 |
| (ANT-1) | ophthalmoplegia | |
| Alpha-1-antitrypsin (AAT) | Hereditary emphysema or | NP_000286.3, NP_001002235.1, |
| | Alpha-1-antitrypsin deficiency | NP_001002236.1, NP_001121172.1, |
| | | NP_001121173.1, NP_001121174.1, |
| | | NP_001121175.1, NP_001121176.1, |
| | | NP_001121177.1, NP_001121178.1, |
| | | NP_001121179.1, AAA51546.1, |
| | | AAB59375.1 |
| Aquaporin 1 (AQP1) | Radiation Induced Xerostomia | NP_932766.1 |
| | (RIX) | NP_001126220.1 |
| | | AAH22486.1 |
| ATPase copper transporting alpha | Menkes syndrome | NP_000043.4 |
| (ATP7A) | | NP_001269153.1 |
| ATPase, Ca++ transporting, | Chronic heart failure | NP_001672.1, NP_733765.1 |
| cardiac muscle, slow twitch 2 | | |
| (SERCA2) | | |
| C1 esterase inhibitor (C1EI) | Hereditary Angioedema (HAE) | NP_000053.2 |
| | | AAH11171.1 |
| | | AAB59387.1 |
| | | AAA35613.1 |
| Cyclic nucleotide gated channel | Achromatopsia (ACHM) | NP_001073347.1 |
| alpha 3 (CNGA3) | | AF272900.1 |
| | | AAH96300.1 |
| | | AAI50602.1 |
| Cyclic nucleotide gated channel | Achromatopsia (ACHM) | NP_061971.3 |
| beta 3 (CNGB3) | | AAF86274.1 |
| Cystic fibrosis transmembrane | Cystic fibrosis | NP_000483.3 |
| conductance regulator (CFTR) | | |
| Galactosidase, alpha (AGA) | Fabry disease | NP_000160.1 |
| Glucocerebrosidase (GC) | Gaucher disease | NP_000148.2, NP_001005741.1, |
| | | NP_001005742.1, NP_001165282.1, |
| | | NP_001165283.1 |
| Granulocyte-macrophage | Prostate cancer | NP_000749.2 |
| colonystimulating factory | | |
| (GM-CSF) | | |
| HIV-1 gag-proΔrt (tgAAC09) | HIV infection | SEQ ID NOs. 1-5 of |
| | | WO2006073496 |
| Lipoprotein lipase (LPL) | LPL deficiency | NP_000228.1 |
| Medium-chain acyl-CoA | Medium-chain acyl-CoA | NP_000007.1, NP_001120800.1, |
| dehydrogenase (MCAD) | dehydrogenase (MCAD) deficiency | NP_001272971.1, NP_001272972.1, |
| | | NP_001272973.1 |
| Myosin 7A (MYO7A) | Usher syndrome 1B | NP_000251.3, NP_001120651.2, |
| | | NP_001120652.1 |
| Poly(A) binding protein nuclear 1 | Oculopharyngeal Muscular | NP_000321.1 |
| (PABPN1) | Dystrophy (OPMD) | |
| Propionyl CoA carboxylase, alpha | Propionic acidaemias | NP_000273.2, NP_001121164.1, |
| polypeptide (PCCA) | | NP_001171475.1 |
| Rab escort protein-1 (REP-1) | Choroideremia (CHM) | NP_001138886.1 |
| | | NP_001307888.1 |
| | | CAA55011.1 |
| Retinal pigment | Leber congenital amaurosis | NP_000320.1 |
| epithelium-specific protein 65 kDa | | |
| (RPE65) | | |
| Retinoschisin 1 (RS1) | X-Linked Retinitis Pigmentosa | NP_000321.1 |
| | (XLRP) | |
| Short-chain acyl-CoA | Short-chain acyl-CoA | NP_000008.1 |
| dehydrogenase (SCAD) | dehydrogenase (SCAD) deficiency | |
| Very long-acyl-CoA | Very long-chain acyl-CoA | NP_000009.1, NP_001029031.1, |
| dehydrogenase (VLCAD) | dehydrogenase (VLCAD) | NP_001257376.1, NP_001257377.1 |
| | deficiency | |

The embodiments of the present invention have been described above, but the present invention is not limited thereto, and those skilled in the art can understand that modifications and changes can be made within the scope of the purport of the present invention. The manner of modifications and changes should fall within the scope of protection of the present invention.

1. A variant of native AAV 8 or AAV9 comprising a substituted amino acid sequence relative to native AAV 8 or AAV9 capsid protein, the substituted amino acid sequence is located at VR VIII region of the native AAV 8 or AAV9 capsid protein, the native AAV 8 is with an amino acid sequence of SEQ ID NO:1, the native AAV 9 is with an amino acid sequence of SEQ ID NO:43.

2. The variant AAV capsid protein of claim 1, wherein the substituted amino acid sequence is located at amino acid position 585 to 597 or 585 to 598 of SEQ ID NO:1; or at the amino acids corresponding to amino acid position 583 to 595 or 583 to 596 of SEQ ID NO:43.

3. The variant AAV capsid protein of claim 2, wherein the substituted sequence located at the position amino acids 585 to 598 of SEQ ID NO:1 is:
X₁X₂X₃X₄X₅X₆X₇X₈X₉X₁₀X₁₁X₁₂X₁₃X₁₄, wherein Formula I:
X₁ is Asn, or Tyr,
X₂ is Leu, or Asn, or Gln, or Lys, or His, or Phe,
X₃ is Gln, or Asn,
X₄ is Gln, or Asn, or Ser, or Ala, or Asp, or Gly,
X₅ is Gln, or Thr, or Ala, or Gly, or Ser, or Asn,
X₆ is Asn, or Ala, or Ser, or Asp, or Thr, or Gln,
X₇ is Thr, or Ser, or Ala, or Arg, or Glu, or Gly,
X₈ is Ala, or Gln, or Asp, or Gly, or Arg, or Thr,
X₉ is Pro, or Ala, or Thr,
X₁₀ is Gln, or Thr, or Ala, or Ile, or Ser, or Asp,
X₁₁ is Ile, or Ala, or Thr, or Val, or Thr, or Ser, or Tyr
X₁₂ is Gly, or Gln, or Ser, or Ala, or Glu,
X₁₃ is Thr, or Ala, or Leu, or Asp, or Ser, or Asn, or Val, or Trp, or Met,
X₁₄ is Val, or Asp,
the sequence doesn't comprise an amino acids sequence of SEQ ID NO:2.

4. The variant AAV capsid protein of claim 2, wherein the substituted sequence located at the position amino acids 585 to 597 of SEQ ID NO:1 is:
X₁X₂X₃X₄X₅X₆X₇X₈X₉X₁₀X₁₁X₁₂X₁₃, wherein Formula II:
X₁ is Asn,
X₂ is Leu, or Asn, or Phe,
X₃ is Gln,
X₄ is Gln, or Asn, or Ser, or Ala,
X₅ is Thr, or Ala, or Ser,
X₆ is Asn, or Ser, or Thr,
X₇ is Thr, or Ala, Gly,
X₈ is Ala, or Gln, or Gly, or Arg,
X₉ is Pro, or Ala,
X₁₀ is Gln, or Ala, or Ile,
X₁₁ is Thr, or Val,
X₁₂ is Gly, or Gln,
X₁₃ is Thr, or Leu, or Asn, or Asp.

5. The variant AAV capsid protein of claim 2, wherein the substituted sequence comprises an amino acid sequence selected from the group consisting of SEQ ID NO:3-43.

6. The variant AAV capsid protein of claim 2, wherein the substituted sequence comprises an amino acid sequence selected from the group consisting of SEQ ID NO:21, SEQ ID NO:25, SEQ ID NO:9, SEQ ID NO:37.

7. The variant AAV capsid protein of claim 2, wherein the substituted sequence located at the position amino acids 583 to 596 of SEQ ID NO:43 is:
X₁X₂X₃X₄X₅X₆X₇X₈X₉X₁₀X₁₁X₁₂X₁₃X₁₄, wherein Formula I:
X₁ is Asn, or Tyr,
X₂ is Leu, or Asn, or Gln, or Lys, or His, or Phe,
X₃ is Gln, or Asn,
X₄ is Gln, or Asn, or Ser, or Ala, or Asp, or Gly,
X₅ is Gln, or Thr, or Ala, or Gly, or Ser, or Asn,
X₆ is Asn, or Ala, or Ser, or Asp, or Thr, or Gln,
X₇ is Thr, or Ser, or Ala, or Arg, or Glu, or Gly,
X₈ is Ala, or Gln, or Asp, or Gly, or Arg, or Thr,
X₉ is Pro, or Ala, or Thr,
X₁₀ is Gln, or Thr, or Ala, or Ile, or Ser, or Asp,
X₁₁ is Ile, or Ala, or Thr, or Val, or Thr, or Ser, or Tyr
X₁₂ is Gly, or Gln, or Ser, or Ala, or Glu,
X₁₃ is Thr, or Ala, or Leu, or Asp, or Ser, or Asn, or Val, or Trp, or Met,
X₁₄ is Val, or Asp,
the sequence doesn't comprise an amino acids sequence of SEQ ID NO:33.

8. The variant AAV capsid protein of claim 2, the substituted sequence located at the position amino acids 583 to 595 of SEQ ID NO:43 is:
X₁X₂X₃X₄X₅X₆X₇X₈X₉X₁₀X₁₁X₁₂X₁₃, wherein Formula III:
X₁ is Asn,
X₂ is Leu,
X₃ is Gln,
X₄ is Asn, or Ser,
X₅ is Ala, or Ser, or Gly,
X₆ is Asn,
X₇ is Thr
X₈ is Ala, or Gln, or Gly,
X₉ is Pro, or Ala,
X₁₀ is Gln, or Thr, or Ala,
X₁₁ is Thr,
X₁₂ is Gly, or Gln, or Ala, or Glu,
X₁₃ is Thr, or Asn, or Asp.

9. The variant AAV capsid protein of claim 7, wherein the substituted sequence comprises an amino acid sequence selected from the group consisting of SEQ ID NO:57-63.

10. The variant AAV capsid protein of claim 7, wherein the sequence comprises an amino acid sequence selected from the group consisting of SEQ ID NO:29, SEQ ID NO:14, SEQ ID NO:9, and SEQ ID NO:11.

11. An isolated polynucleotide comprising a nucleotide sequence that encodes a variant AAV capsid protein of claim 1.

12. A vector comprising the isolated polynucleotide of claim 11.

13. An isolated, genetically modified host cell comprising the polynucleotide of claim 11.

14. A recombinant AAV virion comprising a variant AAV capsid protein of claim 10.

15. A pharmaceutical composition comprising:
a) a recombinant adeno-associated virus virion of claim 14; and
b) a pharmaceutically acceptable excipient.

16. A recombinant AAV vector, comprising polynucleotide encoding a variant AAV capsid protein of claim 10, and an AAV 5′ inverted terminal repeat (ITR), an engineered nucleic acid sequence encoding a functional gene product, a regulatory sequence which directs expression of the gene product in a target cell, and an AAV 3′ ITR.

17. A method of delivering a nucleic acid vector encoding a functional gene product to cells and/or tissues with a recombinant AAV virion of claim 14.

18. A method of treating disease, the method comprising administering to a subject in need thereof an effective amount of a recombinant AAV virion of claim 14, the recombinant AAV virion comprises functional gene product.

19. The method of claim 17, wherein the gene product is a polypeptide.

20. The method of claim 19, wherein the polypeptide is selected from the group consisting of Cystathionine-beta-synthase (CBS), Factor IX (FIX), Factor VIII (F8), Glucose-6-phosphatase catalytic subunit (G6PC), Glucose 6-phosphatase (G6Pase), Glucuronidase, beta (GUSB), Hemochromatosis (HFE), Iduronate 2-sulfatase (IDS), Iduronidase, alpha-1 (IDUA), Low density lipoprotein receptor (LDLR), Myophosphorylase (PYGM), N-acetylglucosaminidase, alpha (NAGLU), N-sulfoglucosamine sulfohydrolase (SGSH), Ornithine carbamoyltransferase (OTC), Phenylalanine hydroxylase (PAH), UDP glucuronosyltransferase 1 family, polypeptide A1 (UGT1A1).

21. The method of claim 20, wherein the recombinant AAV virion comprises a variant AAV8 capsid protein comprising an amino acids sequence selected from the groups consisting of SEQ ID NO:21, SEQ ID NO:25, SEQ ID NO:9, SEQ ID NO:37, or a variant AAV9 capsid protein comprising a amino acids sequence of SEQ ID NO:11.

23. The method of claim 19, wherein the polypeptide is selected from the group consisting of Acid alpha-glucosidase (GAA), ApaLI, Aromatic L-amino acid decarboxylase (AADC), Aspartoacylase (ASPA), Battenin, Ceroid lipofuscinosis neuronal 2 (CLN2), Cluster of Differentiation 86 (CD86 or B7-2), Cystathionine-beta-synthase (CBS), Dystrophin or Minidystrophin, Frataxin (FXN), Glial cell-derived neurotrophic factor (GDNF), Glutamate decarboxylase 1 (GAD1), Glutamate decarboxylase 2 (GAD2), Hexosaminidase A, α polypeptide, also called beta-Hexosaminidase alpha (HEXA), Hexosaminidase B, β polypeptide, also called beta-Hexosaminidase beta (HEXB), Interleukin 12 (IL-12), Methyl CpG binding protein 2 (MECP2), Myotubularin 1 (MTM1), NADH ubiquinone oxidoreductase subunit 4 (ND4), Nerve growth factor (NGF), neuropeptide Y (NPY), Neurturin (NRTN), Palmitoyl-protein thioesterase 1 (PPT1), Sarcoglycan alpha, beta, gamma, delta, epsilon, or zeta (SGCA, SGCB, SGCG, SGCD, SGCE, or SGCZ), Tumor necrosis factor receptor fused to an antibody Fc (TNFR:Fc), Ubiquitin-protein ligase E3A (UBE3A), β-galactosidase 1 (GLB1).

24. The method of claim 23, wherein the recombinant AAV virion comprises a variant AAV9 capsid protein comprising an amino acid sequence selected from the group consisting of SEQ ID NOs:9 and 11.

25. The method of claim 19, wherein the polypeptide is selected from the group consisting of Adenine nucleotide translocator (ANT-1), Alpha-1-antitrypsin (AAT), Aquaporin 1 (AQP1), ATPase copper transporting alpha (ATP7A), ATPase, Ca++ transporting, cardiac muscle, slow twitch 2 (SERCA2), C1 esterase inhibitor (ClEI), Cyclic nucleotide gated channel alpha 3 (CNGA3), Cyclic nucleotide gated channel beta 3 (CNGB3), Cystic fibrosis transmembrane conductance regulator (CFTR), Galactosidase, alpha (AGA), Glucocerebrosidase (GC), Granulocyte-macrophage colonystimulating factory (GM-CSF), HIV-1 gag-proArt (tgAAC09), Lipoprotein lipase (LPL), Medium-chain acyl-CoA dehydrogenase (MCAD), Myosin 7A (MYO7A), Poly(A) binding protein nuclear 1 (PABPN1), Propionyl CoA carboxylase, alpha polypeptide (PCCA), Rab escort protein-1 (REP-1), Retinal pigment epithelium-specific protein 65 kDa (RPE65), Retinoschisin 1 (RS1), Short-chain acyl-CoA dehydrogenase (SCAD), Very long-acyl-CoA dehydrogenase (VLCAD).

26. The method of claim 25, the disease is selected from the group consisting of liver disease, central nervous system diseases, and other diseases.


SEQUENCE LISTING (43 sequences)

SEQ ID NO: 1 - protein, 738 aa, Artificial
  WT AAV8 capsid
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 2 - protein, 13 aa, Artificial
  Synthetic peptide fragment
NLQQQNTAPQIGT

SEQ ID NO: 3 - protein, 13 aa, Artificial
  Synthetic peptide fragment
NNQNTNTAPTAGT

SEQ ID NO: 4 - protein, 13 aa, Artificial
  Synthetic peptide fragment
NLQSANTAPQTQT

SEQ ID NO: 5 - protein, 13 aa, Artificial
  Synthetic peptide fragment
NLQQQNTAPTVGA

SEQ ID NO: 6 - protein, 13 aa, Artificial
  Synthetic peptide fragment
NNQAANTQAQTGL

SEQ ID NO: 7 - protein, 13 aa, Artificial
  Synthetic peptide fragment
NLQSGNTQAATSD

SEQ ID NO: 8 - protein, 14 aa, Artificial
  Synthetic peptide fragment
NQNNGASQTPTASD

SEQ ID NO: 9 - protein, 13 aa, Artificial
  Synthetic peptide fragment
NLQSANTAPQTGT

SEQ ID NO: 10 - protein, 13 aa, Artificial
  Synthetic peptide fragment
NLQQTNSAPIVGA

SEQ ID NO: 11 - protein, 13 aa, Artificial
  Synthetic peptide fragment
NLQSGNTQAATAD

SEQ ID NO: 12 - protein, 13 aa, Artificial
  Synthetic peptide fragment
NLQSSSTDPATGD

SEQ ID NO: 13 - protein, 13 aa, Artificial
  Synthetic peptide fragment
NLQNSNTGPTTGT

SEQ ID NO: 14 - protein, 13 aa, Artificial
  Synthetic peptide fragment
NLQSSNTAPATGT

SEQ ID NO: 15 - protein, 13 aa, Artificial
  Synthetic peptide fragment
NLQSGNTQASTAD

SEQ ID NO: 16 - protein, 13 aa, Artificial
  Synthetic peptide fragment
NNQSANTQAQTGL

SEQ ID NO: 17 - protein, 13 aa, Artificial
  Synthetic peptide fragment
NLQNSNTAASTET

SEQ ID NO: 18 - protein, 13 aa, Artificial
  Synthetic peptide fragment
NLQSANTAPATGT

SEQ ID NO: 19 - protein, 13 aa, Artificial
  Synthetic peptide fragment
NLQQQDTAPIVGA

SEQ ID NO: 20 - protein, 13 aa, Artificial
  Synthetic peptide fragment
NNQNATTAPITGN

SEQ ID NO: 21 - protein, 13 aa, Artificial
  Synthetic peptide fragment
NLQSSTAGPQTQT

SEQ ID NO: 22 - protein, 13 aa, Artificial
  Synthetic peptide fragment
NLQQQNTAPIVGA

SEQ ID NO: 23 - protein, 13 aa, Artificial
  Synthetic peptide fragment
NLQDSNTGPTTGT

SEQ ID NO: 24 - protein, 13 aa, Artificial
  Synthetic peptide fragment
NLQAANTAAQTQV

SEQ ID NO: 25 - protein, 13 aa, Artificial
  Synthetic peptide fragment
NLQSGNTRAATSD

SEQ ID NO: 26 - protein, 13 aa, Artificial
  Synthetic peptide fragment
YLQSGNTQAATSD

SEQ ID NO: 27 - protein, 13 aa, Artificial
  Synthetic peptide fragment
NLQSSNTQAATSD

SEQ ID NO: 28 - protein, 13 aa, Artificial
  Synthetic peptide fragment
NLQRGNRQAATAD

SEQ ID NO: 29 - protein, 13 aa, Artificial
  Synthetic peptide fragment
NLQNSNTGPTTEN

SEQ ID NO: 30 - protein, 13 aa, Artificial
  Synthetic peptide fragment
NKQDSSTQATTAI

SEQ ID NO: 31 - protein, 13 aa, Artificial
  Synthetic peptide fragment
NLQSSAETAETER

SEQ ID NO: 32 - protein, 13 aa, Artificial
  Synthetic peptide fragment
NLQQTNTGPIVGN

SEQ ID NO: 33 - protein, 13 aa, Artificial
  Synthetic peptide fragment
NHQSAQAQAQTGW

SEQ ID NO: 34 - protein, 13 aa, Artificial
  Synthetic peptide fragment
NLQGGNTQAATAD

SEQ ID NO: 35 - protein, 13 aa, Artificial
  Synthetic peptide fragment
NLQQTNGAPIVGT

SEQ ID NO: 36 - protein, 13 aa, Artificial
  Synthetic peptide fragment
NLQSSTAGPQSQT

SEQ ID NO: 37 - protein, 13 aa, Artificial
  Synthetic peptide fragment
NLQNSNTAPSTGT

SEQ ID NO: 38 - protein, 13 aa, Artificial
  Synthetic peptide fragment
NFQSSSTDPATGD

SEQ ID NO: 39 - protein, 13 aa, Artificial
  Synthetic peptide fragment
NFQNNTTAADTEM

SEQ ID NO: 40 - protein, 13 aa, Artificial
  Synthetic peptide fragment
NLQQANTGPIVGN

SEQ ID NO: 41 - protein, 13 aa, Artificial
  Synthetic peptide fragment
NHQSQNTTASYGS

SEQ ID NO: 42 - protein, 13 aa, Artificial
  Synthetic peptide fragment
NLQQQNAAPIVGA

SEQ ID NO: 43 - protein, 736 aa, Artificial
  WT AAV9 capsid
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL